(19) United States
(12) Patent Application Publication
(10) Pub. No.: US 2011/0192397 A1
(43) Pub. Date: Aug. 11, 2011

(54) INHALER

(75) Inventors: Matthew Saskar, Cambridge (GB); Robert May, Hitchin (GB); Alan Tweedie, Chippenham (GB); Paul Hardman, Chippenham (GB)

(73) Assignee: Vectura Delivery Devices Limited, Chippenham (GB)

(21) Appl. No.: 13/122,781

(22) PCT Filed: Oct. 7, 2009
(86) PCT No.: PCT/EP2009/063038

§ 371 Date: Apr. 6, 2011

(30) Foreign Application Priority Data
Oct. 9, 2008 (GB) 0818476.4

(51) Int. Cl. A61M 11/00

(52) U.S. Cl. 128/200.17

(57) ABSTRACT

An inhaler (1) for producing an inhalable aerosol of powdered medicament is disclosed. The inhaler (1) comprises an aerosolising device having a cyclone chamber (45) of substantially circular cross-section, inlet (24) and outlet (25) ports at opposite ends of the chamber (45) for the flow of drug laden air through the chamber (45) between said ports (24, 25) and, a bypass air inlet (46) for the flow of clean air into the chamber (45). The bypass air inlets (46) are configured so that air entering the chamber (45) through said inlet (46) forms a cyclone in the chamber (45) that interacts with the drug laden air flowing between the inlet (24) and outlet (25) ports. The inhaler (1) may have a tapered drug laden air flow conduit (70) to accelerate the flow prior to entry into the chamber (45) and/or an impaction element (81, 84, 92, 105) to deagglomerate drug particles.

The present invention relates to inhalers and, in particular, to inhalers for the delivery of dry powder medicament to the lung.

Oral or nasal delivery of a medicament using an inhalation device is a particularly attractive method of drug administration as these devices are relatively easy for patients to use discreetly and in public. As well as delivering medicament to treat local diseases of the airway and other respiratory problems, they have more recently been used to deliver drugs to the bloodstream via the lungs, thereby avoiding the need for hypodermic injections.

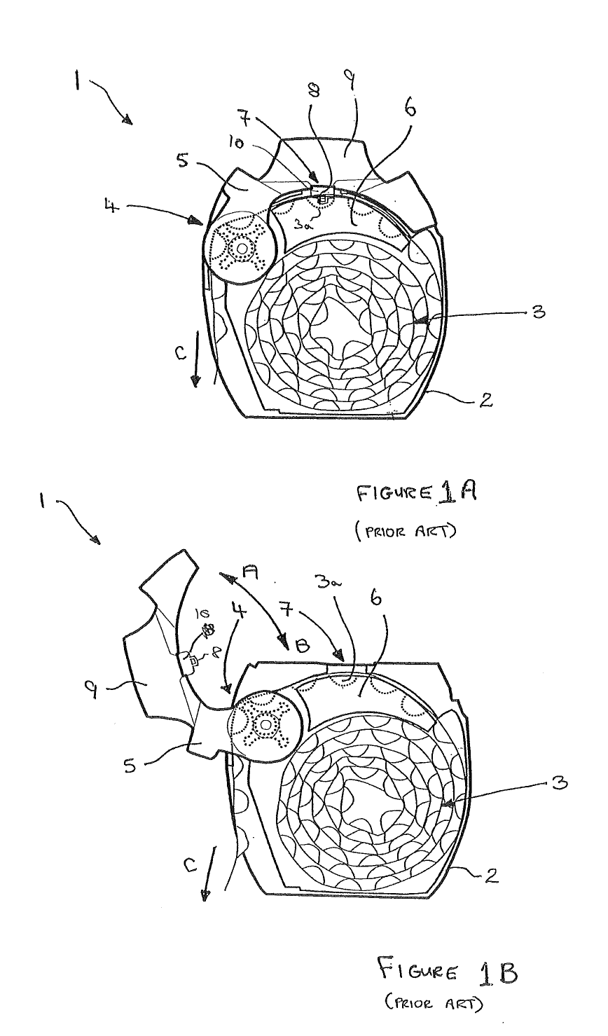

It is desirable to provide an inhaler that is capable of holding a number of individual doses that can be used repeatedly over a period of time without the requirement to open and/or insert a blister or capsule into the device each time it is used. The device known from the Applicant's own earlier application, published as WO 2005/037353A1, addresses this issue by providing a housing that retains a strip of blisters each of which contains a single dose of medicament. When a dose is to be inhaled, an indexing mechanism moves a previously emptied blister away from an opening mechanism so that a fresh one is moved into a position ready to be opened by a piercing element on the device. One embodiment of the device known from this document is described in more detail later, with reference to FIGS. 1A to 1E of the accompanying drawings.

For a medicament in particulate form, the provision of an inhalable aerosol requires an inhaler that can produce a repeatable dose of fine particles. In order for the particles of medicament to reach the deep lung area (alveoli) and thus be absorbed into the bloodstream, the particles must have an effective diameter in the range of approximately 1 to 3 microns. The portion of the emitted aerosol which includes this range of particle size is known as the “fine particle fraction” (FPF). If the particles are larger than 5 microns, they may not be transported by the inhaled airflow deep into the lung, because they are likely to be trapped in the respiratory passages before reaching the deep lung. For example, particles of the order of 10 microns are unlikely to progress further than the trachea and particles of the order of 50 microns tend to deposit on the back of the throat when inhaled. Furthermore, if the particles are less than 1 micron in effective diameter, the particles may not be absorbed into the lung, because they are small enough to be expelled from the lung with the exhaled airflow.

The efficiency of a dry powder inhaler may be measured in terms of the fine particle dose (FPD) or the FPF. The FPD is the total mass of active agent which is emitted from the device following actuation which is present in an aerodynamic particle size smaller than a defined limit. This limit is generally taken to be 5 microns although particles having a diameter less than 3 microns are preferred, for the reasons stated above. The FPD is measured using an impactor or impinger, such as a twin stage impinger (TSI), multi-stage impinger (MSI), Andersen Cascade Impactor (ACI) or a Next Generation Impactor (NGI). Each impactor or impinger has pre-determined aerodynamic particle size collection cut points for each stage. The FPD value is obtained by interpretation of the stage-by-stage active agent recovery quantified by a validated quantitative wet chemical assay where either a simple stage cut is used to determine FPD or a more complex mathematical interpolation of the stage-by-stage deposition is used.

The FPF is normally defined as the FPD divided by the emitted or delivered dose which is the total mass of active agent that is emitted from the device following actuation and does not include powder deposited inside or on the surfaces of the device. The FPF may also, however, be defined as the FPD divided by the metered dose which is the total mass of active agent present in the metered form presented by the inhaler device in question. For example, the metered dose could be the mass of active agent present in a foil blister.

In conventional inhalers, the emitted dose (the amount of medicament that enters the patient's airway) is around 80% to 90% of the dose ejected from the inhaler. However, the FPF may only be around 50% of the emitted dose but the variation in the respirable dose of known inhalers can be +/−20 to 30%. Such variation has historically been acceptable in the case of asthma drugs and the like but regulatory agencies are now requiring much less variability for products for the treatment of respiratory diseases Moreover, it will be appreciated that for the pulmonary delivery of systemic small molecule and protein and peptide drugs or for the administration of drugs such as insulin, growth hormone or morphine, this amount of variation in respirable dose is unacceptable. This is because it is considerably more important to ensure that the patient receives the same intended dose of these types of drugs each time the inhaler is used, so that a predictable and consistent therapeutic effect is achieved with minimal variation from dose to dose. A low respirable dose also means that some of the dose is retained in the blister and this represents a significant wastage of what may be an expensive drug.

It will therefore be appreciated that for systemic and topical pulmonary delivery, the provision of an inhalable aerosol requires an inhaler that can deliver the drug in a highly efficient, accurate and repeatable manner leading to a more predictable and consistent therapeutic effect which minimises any potentially harmful side effects for the patient as well as reducing the amount of costly drug required to deliver a therapeutic dose.

To ensure that a powdered medicament is delivered with an accurately controlled range of particle sizes in order that they are absorbed effectively in the lung, it is necessary to deagglomerate the particles as they flow through the device prior to entry into the patient's airway.

It is known to separate particles of medicament by generating shear forces between the particles, for example by providing a substantial velocity gradient across the particles. One way to achieve this is to provide the inhaler with a cyclone chamber having an axial outlet and a tangential inlet. The drug is entrained in an airflow and allowed to enter the cyclone chamber through the tangential inlet. The high shear forces generated between the particles as they spin around the chamber in the airflow are sufficient to break-up agglomerates of particles before they pass out of the chamber through the outlet. An inhaler having a cyclone chamber is known from the Applicant's own earlier granted European patent No. 1191966 B1. A device for the pulverisation of particles or agglomerates of a powdered inhalation medicament is also known from EPO477222 A1. The device disclosed in this document comprises a rotationally symmetrical vortex chamber with spaced inlet and outlet ports. The inlet ports direct drug laden air into the vortex chamber at a tangent or close to a tangent to the chamber.

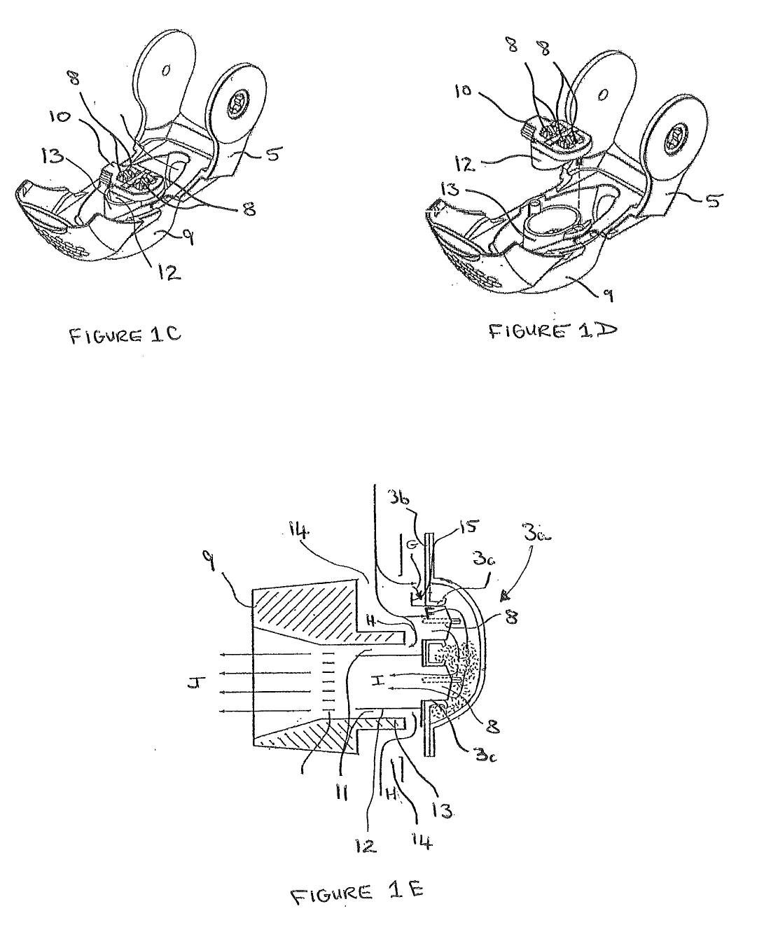

It is also known from the Applicant's co-owned and co-pending European patent application no. 08100886.4 to provide an inhaler which includes an aerosolising device having a generally cylindrical chamber and inlet and outlet ports at opposite ends of the chamber for the flow of drug laden air through the chamber, entering axially at the inlet port and exiting at the outlet port. The inhaler also has a tangential bypass air inlet for the flow of clean, non-drug laden air into the chamber which forms a cyclone in the chamber that interacts with the drug laden air flowing between the inlet and outlet ports. As the bypass air forms a cyclone within the device the drug laden air flow is caused to rotate and follow at least a part helical path towards the outlet port due to the effect of the cyclone upon it. This interaction of the vortex formed from the bypass air spinning around chamber on the drug laden air flowing into the chamber in an axial direction has been found by the Applicant to provide an improvement in performance of the inhaler as the drug laden air is accelerated as it flows through the chamber and experiences increased shear forces and differential velocites which further deagglomerates the particles and improves the fine particle fraction of the emitted dose. An embodiment of the device disclosed in EP08100886.4 is described in more detail below, with reference to FIGS. 2A and 2B of the accompanying drawings.

The present application addresses a number of improvements and modifications to previously disclosed devices and concepts, including those referred to above. For example, one embodiment of the present invention addresses how an inhaler known from WO2005/037353A1 may be modified so as to provide it with an aerosolising device such as that described in EP08100886.4, thereby providing both the functionality and the dose delivery advantages of the inhaler known from WO2005/037353A1 and the cyclone technology described in EP08100886.4. The result is a blister strip type dose inhaler that is simple and intuitive for a patient to use but which also provides an enhanced fine particle fraction of the delivered dose.

According to the invention, there is provided an inhaler for producing an inhalable aerosol of powdered medicament including an aerosolising device having a cyclone chamber of substantially circular cross-section, inlet and outlet ports at opposite ends of the chamber for the flow of drug laden air through the chamber between said ports and, a bypass air inlet for the flow of clean air into the chamber, said bypass air inlet being configured so that air entering the chamber through said inlet forms a cyclone in the chamber that interacts with the drug laden air flowing between the inlet and outlet ports.

Preferably, the bypass air inlet is configured so that bypass air enters the chamber through said bypass air inlet substantially tangential to the wall of the cyclone chamber.

The inhaler may comprise a drug laden air flow conduit that leads to the inlet port and through which drug laden air flows prior to entry into the cyclone chamber.

In one embodiment, the drug laden air flow conduit is at least partially tapered to accelerate the flow in a direction towards the inlet port. The inlet port may alternatively or additionally be offset from the longitudinal axis of the cyclone chamber.

The inhaler may comprise an impaction element in the flow positioned such that at least some drug particles in the drug laden air flow impact the impaction element.

In some embodiments, the impaction element is in the cyclone chamber. Preferably, the impaction element is positioned above the inlet port such that drug particles impact the impaction element after or upon entry into the cyclone chamber.

The impaction element may comprise a plate having an impaction surface that extends in a plane substantially at right-angles to the direction of flow of drug laden air into the chamber through the inlet port. The impaction plate may also extend in a plane at an angle up to about 135 degrees relative to the direction of flow of drug-laden air.

In a preferred embodiment, the plate comprises a blade, the edges of said blade being chamfered, tapered or otherwise shaped so as to minimise disruption to airflow in the chamber. The impaction plate may also be shaped so as to present a convex surface to the flow of drug-laden air.

If the inlet port to the cyclone chamber is offset, the impaction element preferably extends radially inwardly from the side wall of the chamber above the offset inlet port so that it is located directly within the cyclonic airflow generated from bypass air entering the bypass air inlets.

The impaction element includes an impaction surface against which drug particles impact. Preferably, the impaction surface meets the side wall of the chamber from which it extends in a smooth curve.

The impaction element may be located at the outlet to the cyclone chamber. The outlet port can be formed from a mesh. In this case, an impaction element at the outlet may be formed integrally with the mesh. By arranging the impaction element at the outlet to the cyclone chamber, the particles have had the opportunity to accelerate and reach their maximum possible velocity as they travel through the cyclone chamber prior to impaction. The deagglomerating effects are enhanced if the particles are travelling faster at the point of impaction.

In another embodiment, the inlet port is formed from a deagglomerating mesh so that the drug laden air flows through the mesh into the cyclone chamber.

According to a preferred embodiment of the invention, the inhaler comprises a housing to receive a puncturable blister containing a dose of medicament for inhalation and an actuator pivotally attached to the housing, the actuator having a mouthpiece through which a dose of medicament is inhaled by a user and a blister piercing member, wherein the actuator is pivotable to cause the blister piercing member to puncture the lid of a blister, the cyclone chamber being located in the actuator.

Preferably, the housing is configured to receive a strip of blisters each containing a dose of medicament for inhalation, the actuator also being configured to sequentially move each blister into alignment with the blister piercing member so that the blister piercing member punctures the lid of an aligned blister.

In a preferred embodiment, the inhaler comprises an actuator insert that locates in the mouthpiece, the cyclone chamber and the bypass air inlets being formed by said insert.

The cyclone chamber and the bypass air inlets may comprise a recess. In this case, the actuator includes a plate that locates in the mouthpiece and extends over the insert to close the recess.

In one embodiment, the piercing member is attached to the actuator and extends over the plate. The drug laden air flow conduit can be formed in the piercing member. However, it can also be formed in the piercing member and in a passageway that extends from the piercing member to the inlet port to the cyclone chamber.

The piercing member preferably comprises a body having a first piercing element that extends over the plate and a second piercing member that extends over the aperture in the plate, and the drug laden air flow conduit extends through the piercing member for the flow of drug laden air out of a blister and through the aperture in the plate.

In embodiments where there is a plate extending over the insert, the impaction element may comprise a member extending over the aperture in the plate, the member being supported by legs upstanding from the plate. It is also possible to provide a deagglomerating mesh in the plate.

In some embodiments, the inhaler comprises locating pins on the actuator and cooperating lugs on the insert and the plate to position the insert and the plate within the mouthpiece. Preferably, the piercing member locates on the pins over the insert and the plate to position the piercing member on the actuator.

In one embodiment, the cyclone chamber extends in an axial direction for substantially the entire height of the mouthpiece. However, the actuator may comprise a diffuser at the outlet to the cyclone chamber so that the cyclone chamber does not extend for the full height of the mouthpiece.

In other embodiments, a deaggregating element may be located in the cyclone chamber. The deaggregating element can comprises a plurality of vanes or a bladed element rotatably mounted in the chamber such that it spins when a user inhales on the mouthpiece. Alternatively, the deaggregating element is freely movable within the cyclone chamber. For example, it may be a spherical or multi-faceted ball.

Embodiments of the invention will now be described, by way of example only, with reference to FIGS. 3A to 23 of the accompanying drawings, in which:

FIGS. 1A and 1B are side-sectional views of a conventional inhalation device to show how the blisters of a strip are sequentially moved into alignment with a blister piercing station by movement of an actuator from the position shown in FIG. 1A to the position shown in FIG. 1B which drives an indexing wheel;

FIG. 1C is a perspective view of the actuator of the device shown in FIGS. 1A and 1B showing the internal surfaces, i.e. the surface that faces the housing of the inhaler, more clearly;

FIG. 1D is an exploded perspective view of the actuator shown in FIG. 1C to demonstrate how the piercing head is attached to the actuator;

FIG. 1E is a generalised transverse cross-sectional view through the actuator shown in FIGS. 1C and 1D, when the piercing elements have pierced the lid of a blister, to illustrate the air flow paths through the actuator, piercing head and blister;

FIG. 2A is a cross-sectional side view of a portion of an inhalation device having a bypass air cyclone, as described and illustrated in the Applicant's earlier co-pending application referred to above;

FIG. 2B is a cross-section along the line X-X of the device shown in FIG. 1;

FIG. 3A is a perspective view of an actuator assembly according to an embodiment of the present invention;

FIG. 3B is an exploded perspective view of the actuator assembly shown in FIG. 3A;

FIG. 3C is a longitudinal cross-sectional view taken through the assembled actuator shown in FIG. 3A;

FIG. 3D is a transverse cross-sectional view taken through the assembled actuator shown in FIG. 3A;

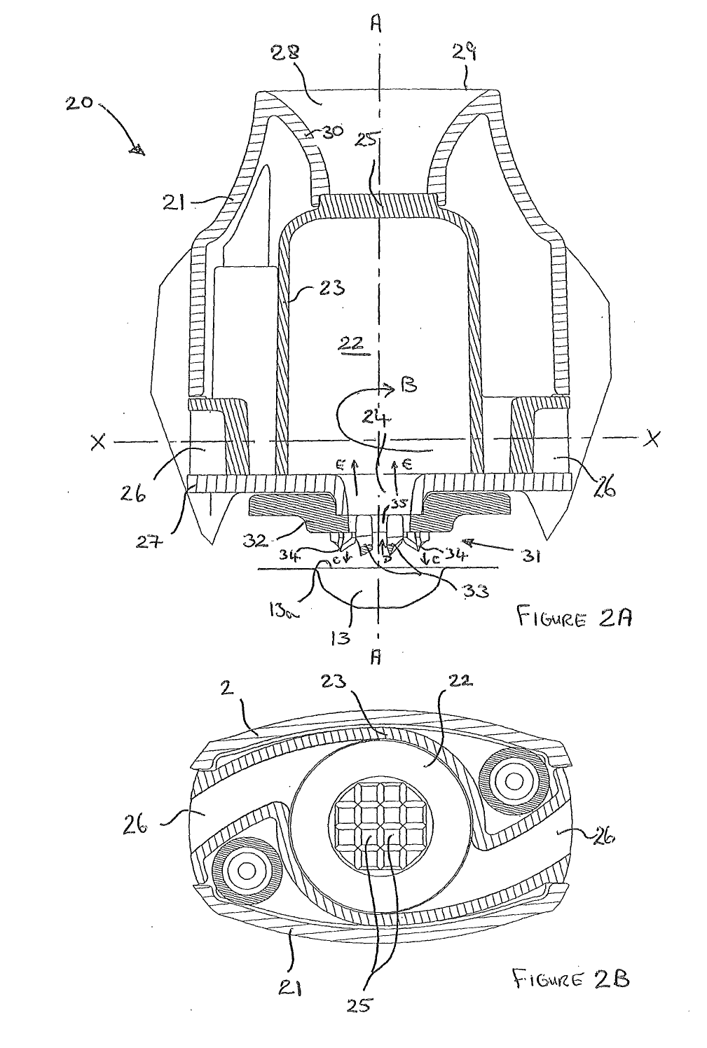

FIG. 4 is a cross-sectional side view of a modified version of the portion of the inhalation device shown in FIG. 2A, according to the present invention;

FIG. 5 is a modified version of the plate used in the embodiment of FIGS. 3A to 3D and embodying one of the concepts shown in FIG. 4;

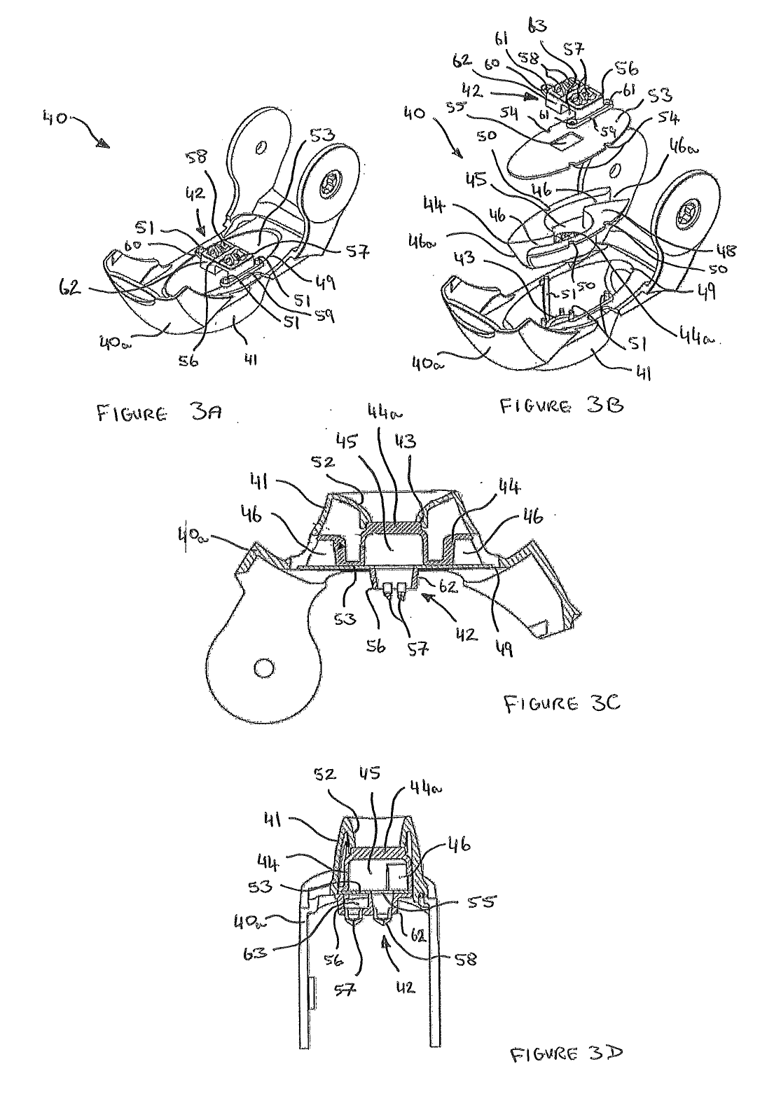

FIG. 6 is a modified version of the insert used in the embodiment of FIGS. 3A to 3D;

FIG. 7A is a perspective view of another modified version of the insert used in the embodiment of FIGS. 3A to 3D;

FIG. 7B is a cross-sectional view of the insert shown in FIG. 7A;

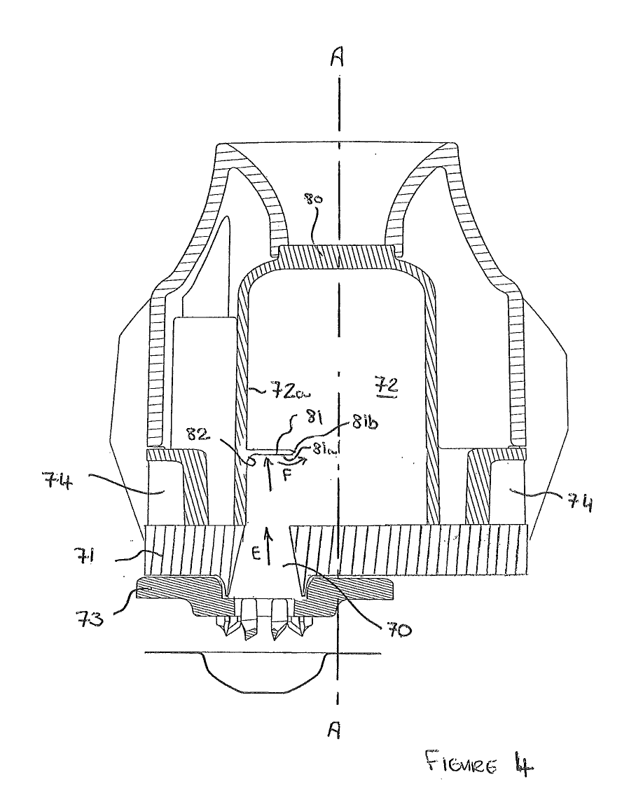

FIG. 8A is a perspective view of a modified version of the piercing head used in the embodiment of FIGS. 3A to 3D;

FIG. 8B is a cross-sectional side view through the piercing head shown in FIG. 8A;

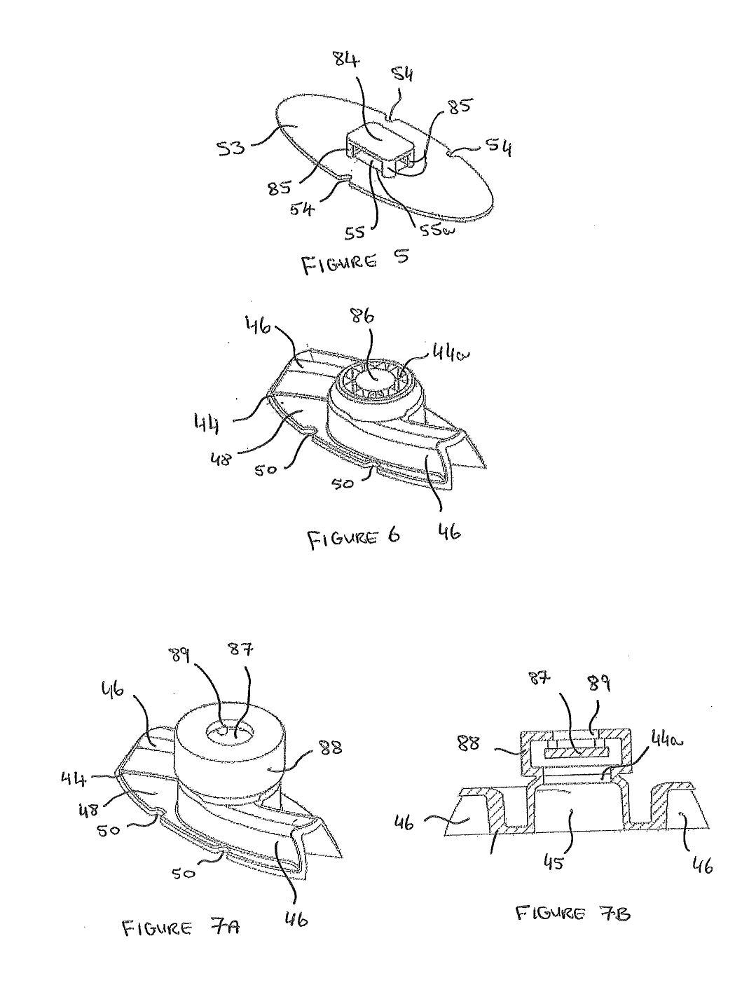

FIG. 9 is another modified version of the plate used in the embodiment of FIGS. 3A to 3D in which the aperture is offset;

FIG. 10 is yet another modified version of the plate used in the embodiment of FIGS. 3A to 3D in which the aperture is offset and includes an impaction element;

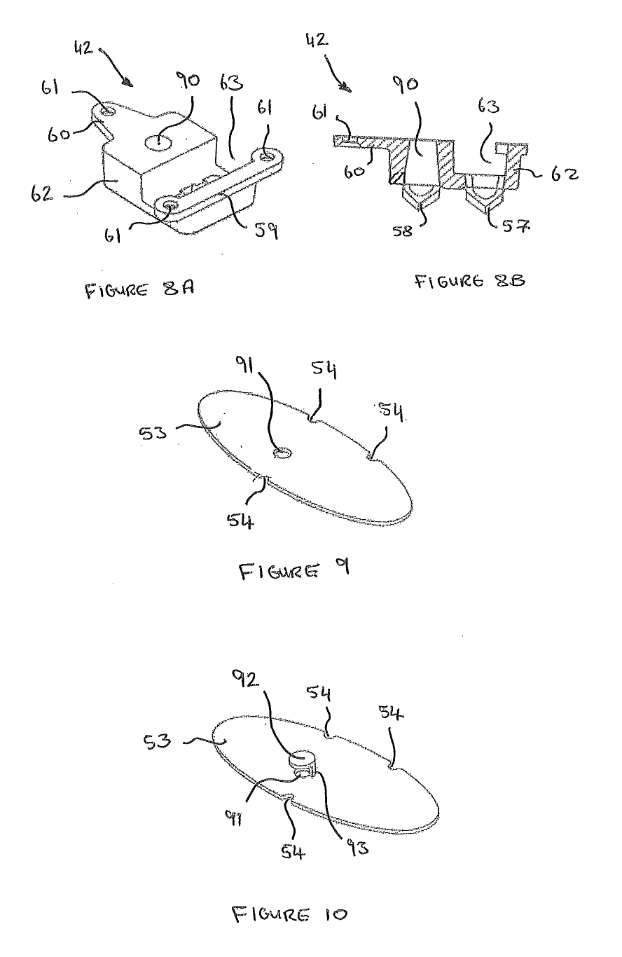

FIG. 11 is another modified version of the insert used in the embodiment of FIGS. 3A to 3D which includes a deaggregating mesh at the outlet to the cyclone chamber;

FIG. 12 is another modified version of the plate used in the embodiment of FIGS. 3A to 3D in which the aperture in the plate is formed from a deaggregating mesh;

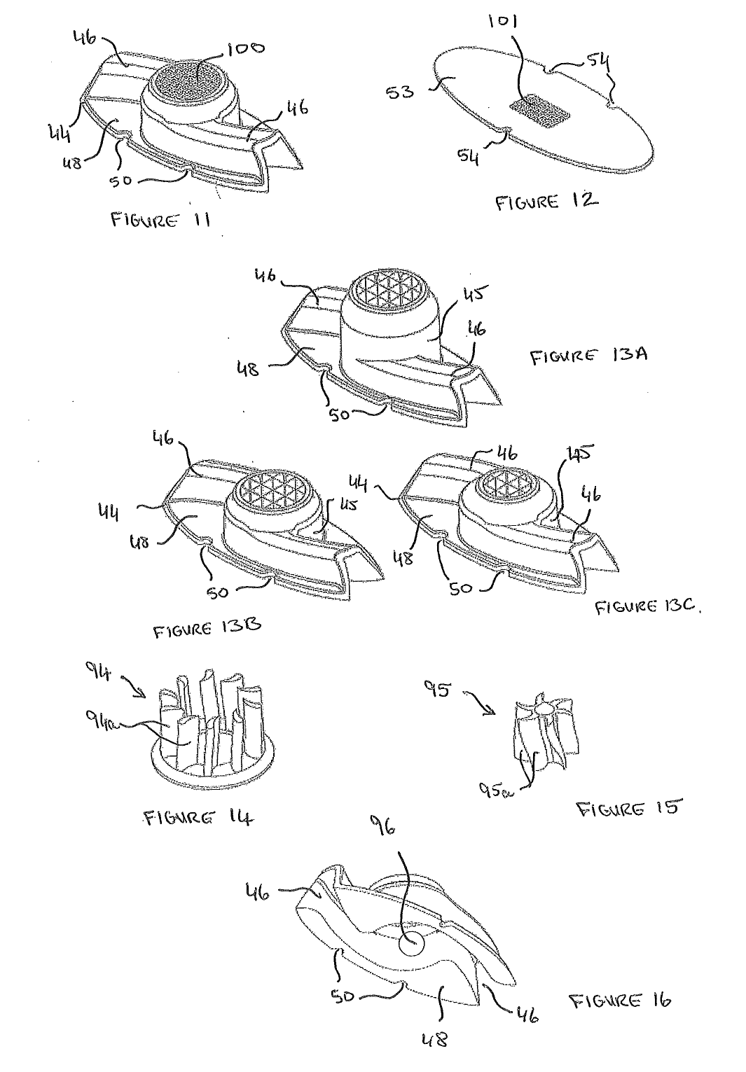

FIGS. 13A to 13C illustrate alternative versions of the insert used in the embodiment of FIGS. 3A to 3D;

FIG. 14 illustrates an insert for a cyclone chamber in the form of a stator;

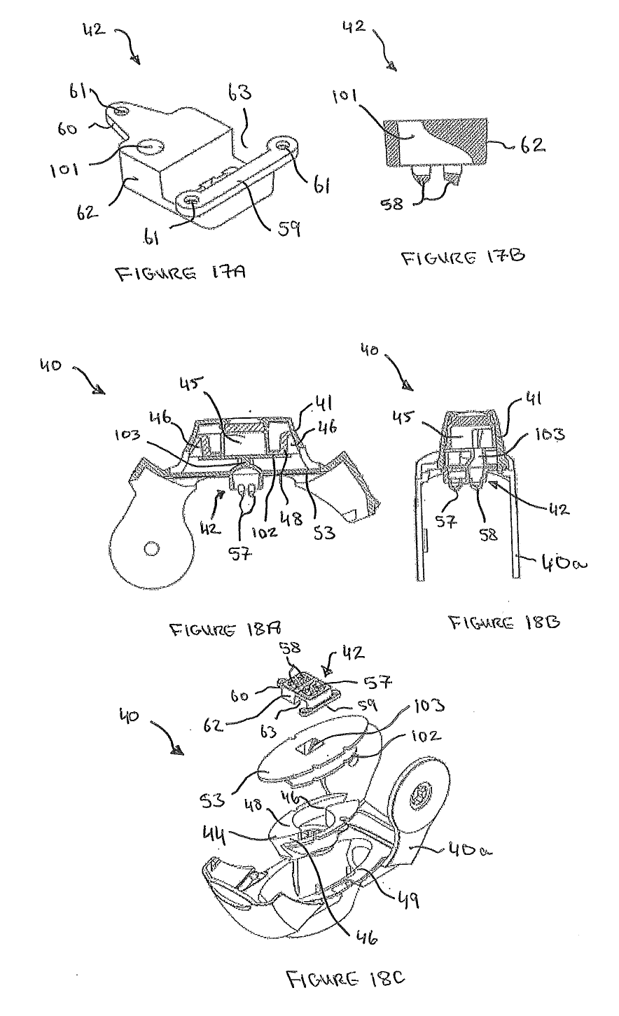

FIG. 15 illustrates an insert for a cyclone chamber in the form of a rotor that is mounted so that it will spin within the cyclone when a patient inhales;

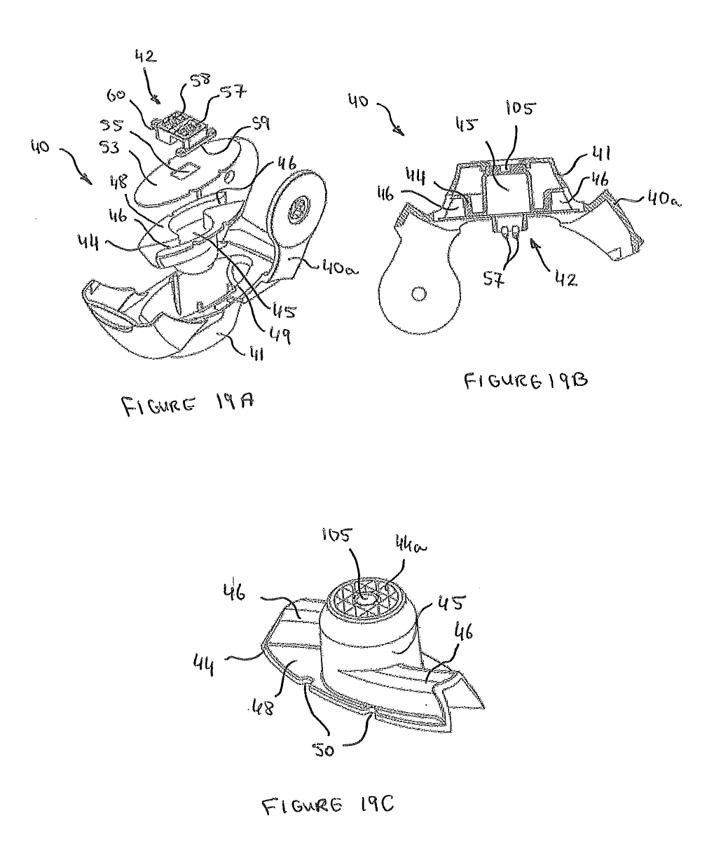

FIG. 16 illustrates how a loose element, such as a ball, may be located in the chamber formed from the insert used in the embodiment of FIGS. 3A to 3D;

FIG. 17A illustrates another modified version of the piercing head used in the embodiment of FIGS. 3A to 3D which has an offset, tapered drug laden air flow path;

FIG. 17B is a cross-sectional side view through the plate shown in FIG. 17A;

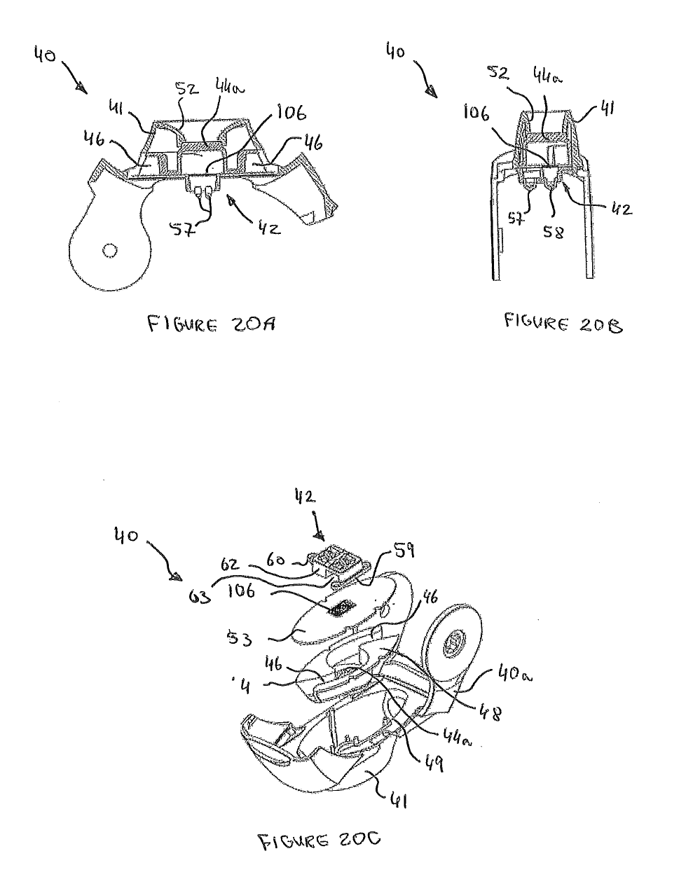

FIG. 18A to 18C illustrate a longitudinal cross sectional, a transverse cross-sectional and an exploded perspective view respectively, of a modified version of the actuator described with reference to FIGS. 3A to 3D which is provided with an elongated, and offset, drug flow path to the cyclone;

FIG. 19A to 19C illustrates an exploded perspective view and a longitudinal cross-sectional view respectively, of another modified version of the actuator described with reference to FIGS. 3A to 3D and in which the diffuser has been omitted, the cyclone chamber lengthened and an impaction element incorporated in the mesh forming the outlet port from the cyclone chamber;

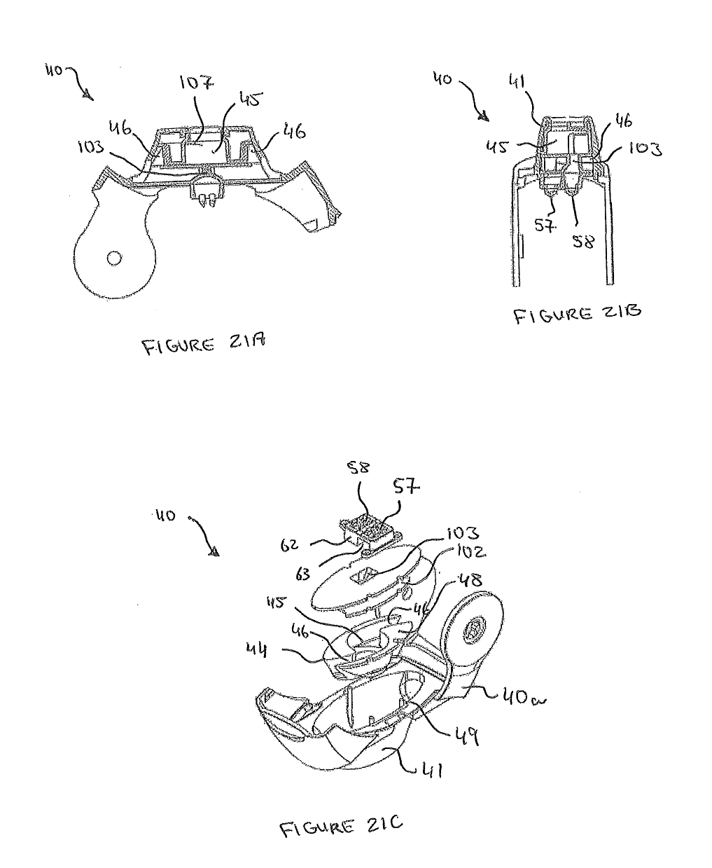

FIG. 20A to 20C illustrate a longitudinal cross-sectional, a transverse cross-sectional and an exploded perspective view respectively, of another modified version of the actuator described with reference to FIGS. 3A to 3D and in which a deaggregation mesh is formed in the aperture in the plate between the piercing head and the insert so that the drug dose passes though said mesh on entry into the cyclone chamber; and

FIG. 21A to 21C illustrate a longitudinal cross-sectional, a transverse cross-section and an exploded perspective view respectively, of another modified version of the actuator described with reference to FIGS. 3A to 3D in which there is an elongated, offset entry to the cyclone and an impaction element in the mesh at the exit to the cyclone chamber; and.

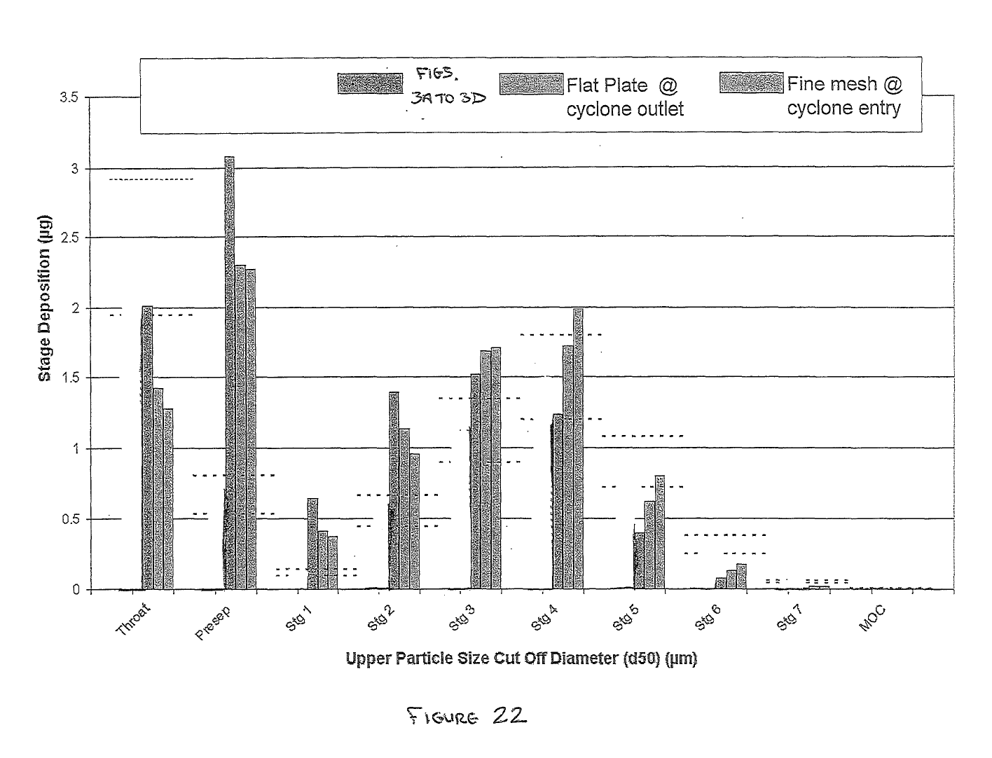

FIG. 22 is a graph to compare deposition relative to various stages of the Next Generation Impactor, showing an increased trend in deposition toward the lower stages.

Referring initially to FIGS. 1A and 1B of the accompanying drawings, there is shown a known inhaler 1 having a housing 2 containing a coiled strip of blisters 3. An indexing mechanism 4 comprising a single actuating lever 5 unwinds the coil 3 one blister at a time so that they pass over a blister locator chassis 6 and successively through a blister piercing station 7, when the actuator 5 is pivoted in a direction indicated by arrow “A” in FIG. 1B. The blister 3a located at the blister piercing station 7 on each movement of the actuator 5 is pierced on the return stroke of the actuator 5 (in the direction indicated by arrow “B” in FIG. 1B) by piercing elements 8 formed on a piercing head 10 mounted to the actuator 5 (see FIG. 1D) so that, when a user inhales through a mouthpiece 9 which is formed integrally with the actuator 5, an airflow is generated within the blister 3a to entrain the dose contained therein and carry it out of the blister 3a via the mouthpiece 9 and into the user's airway.

To reduce the overall pressure drop across the device and make it easier for the patient to inhale a dose, outside air is introduced into the exit airflow through an axially extending bypass conduit 11, as shown most clearly in FIG. 1E. The piercing head 10 has a tubular section 12 which locates within an integrally formed wall 13 upstanding from the actuator 5 within the mouthpiece 9. The bypass conduit 11 is formed from an annular gap between the tubular section 12 and the wall 13, through which bypass air is drawn into the mouthpiece 9 together with the airflow that has passed through the blister 3a. The bypass air that flows along conduit 11 reduces the overall resistance to inspiratory flow, making the device easier to use. As shown in FIG. 1E, when a patient inhales through the mouthpiece 9, air is drawn from outside through holes 14 between the mouthpiece 9 and the actuator 5 from where it flows into a blister 3a through the aperture 3c in the lid 3b, as indicated by arrow marked “F”. In addition to inlet airflow through the aperture 3c, air is also drawn into the blister 3a through the space between the lid 3b of the blister 3a and the surface 15 of the blister piercing head 10, as indicated by arrow marked “G”. In addition to airflow into the blister 3a, air is also drawn through the bypass conduit 11 (in the direction of the arrow marked “H”) and joins the exit airflow leaving the blister 3a through the aperture 3c in the blister lid 3b, in the direction of arrow marked “I”. The dose is entrained in the exit airflow and this airflow from the blister 3a together with the air that has flowed into the mouthpiece 9 via the bypass conduit 11 passes out of the device into the patient's airway, in the direction of arrows marked “J”. It will be noted that the bypass air flowing along bypass conduit 11 is travelling in the same direction as the drug laden air leaving the blister 3a. Therefore, the bypass air has little or no effect on the drug laden air and serves primarily to reduce the pressure drop across the device to make it easier for the patient to inhale.

Various modifications to the device shown in FIGS. 1A to 1E have also been proposed. For example, in the Applicant's own co-pending European application No. 07111998.6, the device has been modified so that all the used blisters are retained within the device so that the patient does not come into contact with the used blisters. In one embodiment described in this previously filed application, a spiral wound element is provided within the housing to receive the used portion of the blister strip and coil it up within the housing. Furthermore, a dividing wall may be provided to separate the housing into unused and used blister compartments so as to minimise any possible contact of the unused blisters with residual drug. Despite these modifications, the device still has the actuator to sequentially index the blister strip and cause a blister piercing element to pierce the lid of an aligned blister and so the modifications proposed herein are equally applicable to these versions of the device.

Referring now to FIG. 2A, there is shown a portion of another inhalation device 20, as described and illustrated in the Applicant's own earlier co-pending application, which modifies the bypass air flow so that it does more than simply reduce the pressure drop across the device but also assists in deagglomeration of the drug dose. With reference to FIG. 2A, the device has a mouthpiece 21 defining an internal chamber 22 having a chamber wall 23, a drug laden air inlet port 24, an outlet port 25 and bypass air inlets 26. A cross-sectional view taken along the line X-X in FIG. 2A is also shown in FIG. 2B.

The device 20 includes a base 27 extending across a lower end of the mouthpiece 21 and closing the chamber 22. The drug laden air inlet port 24 is formed in, and extends through, the base 27 and is coaxial with the longitudinal axis (A-A in FIG. 2A) of the chamber 22.

Although the base 27 could be formed integrally with the mouthpiece 21, it is preferably formed as a separate component which is attached to the mouthpiece 21 or to the end of the chamber 22 during assembly.

As shown in FIG. 2B, the bypass or clean, non-drug laden air inlets 26 are preferably tangentially oriented arcuately shaped channels formed in the sides of the mouthpiece 21 and the base 27 forms the lowermost wall and encloses the lower end of the chamber 22 (apart from the drug laden air inlet port 24), but also forms the lower surface of the channels 26 so that the channels 26 are open only at each of their ends. Although two channels are shown in the present embodiment, it will be appreciated that one channel is sufficient.

As the bypass air inlets 26 are arranged tangentially or so as to direct the bypass air in a substantially tangential direction into the chamber 22, the clean air flowing through these inlets 26 into the chamber 22 is forced to spin around the chamber 22 so as to form a cyclone or vortex (as indicated by arrow “B” in FIG. 2A).

The outlet port 25 may be in the form of a mesh extending across the end of the chamber 22 through which the entrained drug may flow out of the chamber 22 into the patient's airway. Preferably, the mouthpiece 21 incorporates a flow diffuser 28 that extends beyond the outlet port 25 and has a cross-sectional area that gradually increases towards the top edge 29 of the mouthpiece 21. The walls 30 of the diffuser 28 in this region may be curved in shape.

A piercing device 31 is disposed beneath the mouthpiece 21 on the opposite side of the base 27 and may extend from or be connected to the base 27. As can most clearly be seen from FIG. 2A, the piercing device 31 comprises a piercing head 32 having piercing elements 33,34 depending there from. The blister piercing elements 33,34 are configured to puncture the lid 3b of a blister 3a so that, when a patient inhales through the mouthpiece 21, clean air enters the blister 3a through the air inlet flow passages formed by blister piercing elements 34 (in the direction of arrow “C” in FIG. 2A) and entrains the dose contained in the blister 3a. The drug laden air then flows out of the blister 3a through a central drug laden air outlet passage 35 (in the direction of arrow “D”). The drug laden air outlet passage 35 is connected to the drug laden air inlet port 24 of the chamber 22 so that it flows in an axial direction into the chamber 22 (in the direction indicated by arrow “E”). At the same time, clean bypass air enters the chamber 22 through the tangential bypass air inlets 26 and spins around the chamber 22 (in the direction of arrow “B”) forming a vortex or cyclone.

An embodiment of the present invention is illustrated in FIGS. 3A to 3D. In this embodiment, the bypass air cyclone concepts described in EP08100886.4 are combined with the actuator of the inhalation device described above and shown in FIGS. 1A to 1E. This is achieved by modifying the actuator so as to enable it to incorporate a small bypass air cyclone within the confines of the mouthpiece.

The overall outward appearance of the actuator 40 of the embodiment of FIGS. 3A to 3D remains largely unchanged to the embodiment of FIG. 1C to 1E and still includes a mouthpiece 41 that is integrally formed with the main body 40a of the actuator 40. However, the blister piercing head 42 no longer has a tubular portion 12 that is received concentrically within an integrally formed wall 13 within the mouthpiece 9. Instead, the actuator 40 has a seat 43 on which is mounted a moulded insert 44 that is wholly received within the space defined within the mouthpiece 41. The moulded insert 44 defines a cylindrical cyclone chamber 45 with arcuate tangential bypass air passages 46 leading from opposite ends 46a of the insert 44 to the chamber 45. The upper end of the insert 44 (the end furthest away from the piercing head 42) is closed apart from a mesh 44a formed at the outlet to the cyclone chamber 45 whereas the bottom end of the insert 44 (the end closest to the piercing head 42) is open so that the cyclone chamber 45 and the bypass air passages 46 are open along the lower face of the insert 44. The insert 44 is integrally moulded together with a generally oval-shaped flange 48 which is only slightly smaller than an oval shaped opening 49 which is formed where the mouthpiece 41 meets the body 40a of the actuator 40 so that the flange 48 substantially fills the opening 49 when received within the mouthpiece 41. Lugs 50 are provided on the edge of the flange that locate around pins 51 upstanding from the edge of the opening 49 to receive and locate the insert 44 within the mouthpiece 41. When the insert 44 is located within the mouthpiece 41, the ends 46a of each of the bypass air passages 46 are close to the bypass air inlet openings 14 in the actuator 40.

As can be seen most clearly from FIGS. 3C and 3D, the seat 43 to mount the insert 44 is formed at the base of a diffuser defined by a generally curved, preferably arcuate wall 52. It will be appreciated that in order to fit the insert 44 within the confines of the space formed in the mouthpiece 41, the axial length of the cyclone chamber 45 is relatively short and that the height of the bypass air inlet passages 46 are the same or only slightly shorter than the axial length of the cyclone chamber 45. However, it will be appreciated that the dimensions of the bypass air inlet passages 46 may be varied relative to the axial length of the bypass cyclone chamber 45, as will become described later, with reference to FIGS. 13A to 13C. It is also envisaged that the diffuser 52 can be omitted altogether so that the cyclone chamber 45 can be extended so that its axial length is substantially the same as the full height of the mouthpiece 41.

Referring once again to FIGS. 3A to 3D, it can be seen that the open lower end of the cyclone chamber 45 and bypass air flow passages 46 are closed by an oval shaped plate 53 which substantially corresponds in size and shape to the flange 48 of the insert 44. The plate also has lugs 54 that locate around the pins 51 to secure the plate 53 in position so that it extends across the opening 49 and over the insert 44. An aperture 55 is formed through the plate 53 directly beneath the cyclone chamber 45.

The piercing head 42 sits on top of the plate 53 and comprises a body 56 with first and second sets of piercing elements 57, 58. Tabs 59, 60 extend from a lower edge of each side of the body 56 in which holes 61 are formed. The upper ends of each of the pins 51 extend through the holes 61 to locate the body 56 on the plate 53 so as to attach the piercing head 42 to the actuator 40.

The body 56 has a peripheral wall 62 which spaces the piercing elements 57,58 away from the plate 53. The first set of piercing elements 57 extend over the plate 53, as can be most clearly seen from FIG. 3D, and an opening 63 is formed in the wall 62 so that, when the blister piercing elements 57, 58 are received within a blister, the first set of piercing elements 57 allow air to flow via said opening 63 and through said piercing elements 57 into the blister.

The second set of piercing elements 58 are positioned over the aperture 55 in the plate and the wall 62 encloses the space between the piercing elements and the plate 53 so that air that has flowed into a blister through the first set of piercing elements 57 and which has entrained a dose contained therein, flows out of the blister via an opening made in the blister by the second set of piercing elements 58 and is directed through the part of the piercing head 42 enclosed by the peripheral wall 62, through the aperture 55 in the plate 53 and into the cyclone chamber 45 where it interacts with clean, non-drug laden air, entering the cyclone chamber 45 through the bypass air passages 46, as has already been explained above with reference to FIGS. 2A and 2B.

It will be appreciated that once insert 44 and the plate 53 have been positioned within the mouthpiece 41, with the lugs 50,54, located around the pins 51 and the top end of the pins 51 passed through the holes 61 in the piercing head 42, the tip of the pins 51 may be deformed by heat or otherwise so as to hold the piercing head 42, the plate 53 and the insert 44 in place within the mouthpiece 41.

Some modifications to the bypass cyclone concepts described above with reference to 2A and 2B have also been proposed all of which have the primary intention of adjusting the particle size distribution of the delivered dose. Some of these will first be considered in general before explaining how the actuator assembly of FIGS. 3A to 3D may be modified to incorporate these general principles in more practical terms.

Turning now to FIG. 4 this illustrates a modified cross-sectional view of the portion of the inhalation device shown in FIG. 2A. In this embodiment, the inlet port 70 in the base 71 is extended so as to form an inlet flow conduit which tapers inwardly towards the chamber 72 in the direction of the drug laden air flow (i.e. in the direction of arrow “E”). Although FIG. 4 shows the tapered flow path or conduit 70 as being formed in the base 71, it will be appreciated that it can alternatively or additionally be formed in the piercing head 73 which is attached on or to the base 71 so as to achieve the same effect. Fundamentally, a tapering drug laden dose flow path 70 ensures that the drug laden air is accelerated as it travels from the blister exit to the entry to the cyclone chamber 72 so that it is travelling faster upon entry into the chamber 72.

Although the tapered drug laden air flow path 70 can be arranged coaxial with the longitudinal axis A-A of the cyclone chamber 72, it is preferable if the drug laden airflow is not coaxial but offset or eccentric from the longitudinal axis of the chamber 72. Most preferably, and as shown in FIG. 4, the inlet port 70 is offset so that it is adjacent to the inner surface 72a of the wall of the chamber 72. As a result, the drug laden air enters the chamber 72 very close to its inner surface 72a and directly interacts with the vortex formed from the bypass air entering the bypass air inlets 74 on entry into the chamber 72. The differential velocities and shear forces are maximised closest to the chamber wall 72a and so the effect of the cyclone as the drug laden air enters the chamber 72 is greatest when the drug laden air inlet port 70 is positioned as close as possible to the side wall 72a of the cyclone chamber 72. It will be appreciated that the drug outlet port 80 remains coaxial with the axis of the cyclone chamber irrespective of whether the drug flow inlet port 70 is offset from the axis.

Although the provision of a tapering, possibly offset, drug inlet flow path may be the only modification, it is alternatively or additionally possible to provide an impaction element. The key benefit of an impaction element is to disaggregate larger drug particles present in the device and so influence the particle size distribution of the dose of drug emitted by the device.

In FIG. 4, an impaction element 81 is shown mounted within the cyclone chamber 72 extending from the side wall 72a directly above the drug laden air flow inlet port 70, so that the drug laden air flow targets the underside of the impaction element 81 (as indicated by arrows “F”). Although some of the smaller particles entering the cyclone chamber 72 will be swept up in the cyclonic bypass air flow prior to reaching the impaction element 81, some of the larger particles will travel in a generally axial direction towards the impaction plate 81 and will impact the underside of the impaction element 81. This assists in deagglomeration of the particles and dislodges drug particles from carrier particles, if present. It also reduces or eliminates the amount of drug that may otherwise travel directly through the chamber 72 between the inlet and outlet ports 70, 80 that would otherwise have little or no interaction with the cyclonic airflow. Consequently, any large drug or carrier particles that would otherwise leave the device instantly are now forced to be involved in the cyclonic airflow.

The impaction element 81 generally takes the form of a flat, concave, convex plate or blade-like member having an underside impaction surface 80a that extends substantially at right angles and radially inwardly from the wall 72a of the cyclone chamber 72 and at right-angles to the direction of drug laden air flow into the chamber 72 from the drug laden air inlet 70. As the impaction element 81 extends into the chamber 72 from its side wall 72a, it is positioned within the vortex created by the bypass air flow where the forces are at their highest and it is expected that this will assists in cleaning off any drug that becomes deposited on the impaction element 81 thereby effectively self-cleaning the impaction element. Angles greater than 90 degrees, up to about 135 degrees, as well as a convex surface presented to the drug-laden air, also reduce the potential for drug to be deposited on the impaction element.

The impaction element 81 may have edges 81b that generally taper towards a pointed tip to create a smoother profile that directs air across its surfaces with minimum resistance and thereby helps prevent drug deposition and also minimises disruption to the cyclonic air flow.

The underside impaction surface 81a preferably has a smooth radiused or curved edge 82 where it meets the chamber wall 16a to minimise particle deposition in this area. The opposite upwardly facing surface of the impaction plate 81 may have a similarly rounded profile although it is acceptable for the impaction plate 81 to meet the chamber wall 72a at a relatively sharp, possibly even 90 degree, angle so as to minimise disruption to the cyclonic airflow passing over the plate 81. However, it is also envisaged that the impaction surface 81a of the plate 81 could present a shaped surface to the impacting airflow. For example, it could have a convex or concave shaped profile with respect to the direction of airflow in the locality of the impaction plate 81. It will also be appreciated that the dimensions of the impaction plate 81 and the open area around the impaction plate 81 through which the drug laden air flow must pass can be varied to alter the effect of the impaction plate on the drug dose.

Although the impaction plate 81 is shown offset from the axis of the chamber 72, it is also envisaged that when the inlet port 70 is coaxial, the impaction element 81 may also be mounted coaxially within the centre of the chamber 72 so as to be positioned directly above the inlet port 70 and so that it doesn't interfere with the cyclonic airflow through the chamber 72. As with the offset plate 81, the edges 81b may be tapered so as to minimise disruption to airflow and deposition.

In FIG. 4, the impaction element 81 is shown positioned at about one third of the height of the chamber 72 from the base 71. However, it will be appreciated that the impaction element 81 can be positioned at any height within the chamber 81 and can also be located at the top of the chamber 72 and/or be formed integrally with a mesh that forms the chamber outlet port 80, as will become apparent from the following description of other embodiments.

Having described the modifications in general terms, reference will now be made to how the embodiments of the present invention shown in FIGS. 3A to 3D may be modified to provide impaction elements and/or tapered flow inlets.

In one embodiment, the impaction element may be positioned at the entry to the cyclone chamber 45 and directly after exiting the blister. Referring to FIG. 5, there is shown a modified version of the plate 53 used in the embodiment of FIGS. 3A to 3D. In this embodiment, an impaction element 84 is spaced a short distance above the drug flow aperture 55 supported by legs 85 that extend upwardly towards the impaction element 84 from the periphery 55a of the aperture 55. It will be appreciated that the impaction element 84 is located within the cyclone chamber 45 when the plate 53 is located on the mouthpiece insert 44.

Alternatively, the impaction element may be located in or close to the cyclone exit. For example, FIG. 6 illustrates a modified version of the insert 44 used in the embodiments of FIGS. 3A to 3D. In this embodiment, an impaction element 86 is formed centrally in the mesh that forms the chamber outlet port 44a.

In the embodiment shown in FIGS. 7A and 7B, a further modification to the insert 44 is shown. The impaction element 87 is positioned above the outlet port 44a, the insert 44 being provided with an additional, cylindrical housing portion 88 that surrounds an impaction plate 87 and has an outlet 89 for the flow of drug laden air out of the housing portion 88 after it has impacted on the impaction plate 87.

As has already been mentioned above, any of the impaction plates described with reference to the embodiments of the present invention may be flat, convex or have concave shaped profile.

Referring now to FIGS. 8A and 8B, there is shown a modified version of the piercing head 42 described with reference to FIGS. 3A to 3D. As can be seen most clearly from FIG. 8B, the flow path 90 that extends through the body 56 from the blister piercing elements 58 to the aperture 55 in the plate 53 is tapered in a direction towards the plate 53 so that the drug laden air flow is accelerated prior to it passing into the cyclone chamber 45. The piercing head 42 may also be modified so as to increase the length of the flow path 90 to allow the drug particles additional time to speed up to the airflow velocity.

FIG. 9 shows another modified version of the plate 53 as used in the embodiment of FIGS. 3A to 3D. In this embodiment, the plate 53 has a smaller aperture 91 that is offset so that the drug laden air will enter the chamber 45 closer to its side wall.

FIG. 10 shows yet another modified version of the plate 53 as used in the embodiment of FIGS. 3A to 3D. In this embodiment, the opening 91 is offset, as in FIG. 9, but an impaction element 92 is spaced from the opening 91 by a support 93 upstanding from part of the periphery of the opening 91 so that drug particles passing through the opening 91 into the cyclone chamber 45 will directly impact the underside of the impaction element 92.

It has also been found that a fine mesh in the drug path can further disaggregate the drug particles. In the embodiment shown in FIG. 11, the insert 44 used in the embodiment of FIGS. 3A to 3D, a fine mesh 100 is located across the exit to the cyclone chamber. The mesh may have a pore size of less than 250 microns or be in a range between 30 and 150 microns. In particular embodiments, the mesh may for example be fine (200 μm aperture, 125μ wire diameter) or coarse (500μm aperture, 160 μm wire diameter).

Alternatively, as shown in FIG. 12, a mesh 101 can form the aperture in the plate 53 so that the drug dose has to pass through it on entry into the cyclone chamber 45. The dimensions of the mesh can be varied to alter aperture size and overall percentage open area to control the extent of the deagglomeration. However, in a preferred embodiment, the aperture is a square of between 0.2 mm and 0.5 mm wide, and the diameter of the bars is between 0.1 mm and 0.2 mm.

As has already been mentioned above, it is possible to modify the size of the cyclone chamber 45 by altering its height, diameter, inlet cross-sectional area and exit cross-sectional area, so as to change the particle size distribution of the emitted dose. Possible modified versions of the insert 44 described with reference to FIGS. 3A to 3D are shown in FIGS. 13A to 13D. In FIG. 13A, the chamber is of maximum axial length and is intended for use in an actuator having no diffuser. FIG. 13B shows an insert 44 having a chamber 45 which is shorter in length and has a relatively large diameter outlet mesh 44a. The insert 44 of FIG. 13C is the same as FIG. 13B, except that the outlet mesh 44a is of a smaller diameter relative to the diameter of the chamber 45.

It has also been found that drug disaggregation may also be increased by increasing the air flow turbulence particle interactions in the cyclone chamber. For example, a fixed or moving element may be introduced into the chamber such as a stator 94 having airflow vanes 94a, as shown in FIG. 14, a spinning rotor 95 having shaped blades 95a, such as that shown in FIG. 15 or, a freely moving element such as a spherical or faceted ball 96, as shown in FIG. 16.

It will be appreciated that maximum effect can be obtained by combining two or more of the embodiments described above with reference to FIGS. 4 to 16.

A further modified version of the piercing head 42 used in the embodiment of FIGS. 3A to 3D is shown in FIG. 17A. It can be seen that the flow path 101 is both tapered and offset so that the drug laden air will enter the chamber 45 closer to the side wall of the chamber 45 and away from its longitudinal axis.

FIGS. 18A to 18C show a modified version of the embodiment of FIGS. 3A to 3D in which the drug flow path from the blister piercing head 42 to the cyclone chamber 45 is elongated so that the drug travels further between the blister and the cyclone chamber 45 and its cross-sectional area reduces towards the cyclone chamber 45 so as to accelerate the flow. The drug flow path is also shown offset from the longitudinal axis of the cyclone chamber 45. As can be seen from FIGS. 18A to 18C, this is achieved by removing the diffuser 52 so that the insert 44 can be moved further into the mouthpiece 41 to leave additional space between the piercing elements 57,58 and the inlet port to the cyclone chamber 44. As shown in FIG. 18, the flange 48 on the insert is spaced from the plate 53 and so an intermediate plate 102 is positioned on the insert 44 so as to close the bypass air passageways 46 and provide an inlet into the cyclone chamber 45. A conduit 103 extends between the intermediate plate and the plate 53 to provide an elongated drug flow path. The conduit 103 is tapered and offset from the longitudinal axis of the cyclone chamber 45. The blister piercing head 42 is positioned over the plate 53 in the usual way and also has a tapered and offset flow path (as shown in the embodiment of FIGS. 17A and 17B) extending through it that meets the tapered and offset flow path formed by the conduit 102, thereby providing an elongated drug flow path between the blister and the cyclone chamber 45.

FIGS. 19A to 19C show yet another modified version of the embodiment shown in FIGS. 3A to 3D. In this embodiment, the diffuser 52 has been removed and the cyclone 45 has been enlarged so that it effectively extends for the full height of the mouthpiece 41. An impaction element 105 is formed together with the insert 44 at the drug outlet 44a from the cyclone 45.

FIGS. 20A to 20C show another modified version of the embodiment shown in FIGS. 3A to 3D. In this embodiment, a disaggregation mesh 106 is formed in the plate 53 so that the drug laden air passes through the mesh on exit from the piercing head 42 and as it enters the cyclone chamber 45. As with the previous embodiment, an impaction plate 105 may be provided at the exit to the cyclone chamber 45.

FIGS. 21A to 21C shows another modified version of the embodiment shown in FIGS. 3A to 3D. This embodiment is similar to the embodiment of FIGS. 18A to 18C in that it has an elongated flow path provided by conduit 103. However, it is also provided with an offset impaction plate 107 extending from the wall of the cyclone chamber 45 at the exit from the cyclone chamber 45.

FIG. 22 is a graph to compare deposition relative to particle diameters using a multi-stage impinger having pre-determined aerodynamic particle size collection cut points for each stage, for the embodiments described with reference to FIGS. 3A to 3D, a device having a flat impaction plate at the exit of the cyclone and a device having a fine deagglomerating mesh at the entry to the cyclone, respectively. From a consideration of this graph, it will be appreciated that an impaction plate disposed at the cyclone outlet port or a fine mesh at the cyclone inlet port help to shift the particle size distribution towards the lower stages resulting in greater lung deposition.

A variety of medicaments may be administered alone by using inhalers of the invention. Such medicaments include those that are suitable for the treatment of asthma, chronic obstructive pulmonary diseases (COPD), respiratory infections, rhinitis, allergic rhinitis, nasal diseases and disorders; general and specific conditions, and systemic diseases with the lung or nasal cavity as the site of delivery. Such medicaments include, but are not limited to, β₂-agonists, eg carmoterol, fenoterol, formoterol, levalbuterol, pirbuterol, reproterol, metaproterenol, rimiterol, salbutamol, salmeterol, indacaterol, terbutaline, orciprenaline, clenbuterol, bambuterol, procaterol, broxaterol, picumeterol, and bitolterol; non-selective β-stimulants such as ephedrine and isoprenaline; phosphodiesterase (PDE) inhibitors, eg methylxanthines, theophylline, aminophylline, choline theophyllinate, and selective PDE isoenzyme inhibitors, PDE 3 inhibitors, eg milrinone and motapizone; PDE 4 inhibitors, eg rolipram, cilomilast, roflumilast, oglemilast, and ONO 6126; PDE 3/4 inhibitors, eg zardaverine and tolafentrine; inducers of HDAC2 eg theophylline; anticholinergics including muscarinic receptor (M1, M2, and M3) antagonists eg atropine, hyoscine, glycopyrrolate, ipratropium, tiotropium, oxitropium, NVA237, pirenzepine, and telenzepine; mast cell stabilisers, eg cromoglycate and ketotifen; bronchial anti-inflammatory agents, eg nedocromil; steroids, eg beclometasone, dexamethasone, fluticasone, budesonide, flunisolide, rofleponide, triamcinolone, butixocort, mometasone, and ciclesonide; disease modifying agents such as methotrexate, leflunomide, teriflunomide, and hydroxychloroquine; histamine type 1 receptor antagonists, eg cetirizine, loratadine, desloratadine, fexofenadine, acrivastine, terfenadine, astemizole, azelastine, levocabastine, chlorpheniramine, promethazine, cyclizine, and mizolastine; antibacterial agents and agents for cystic fibrosis and/or tuberculosis treatment, eg Pseudomonas aeruginosa infection vaccines (eg Aerugen®), mannitol, denufosol, glutathione, N-acetylcysteine, amikacin duramycin, gentamycin, tobramycin, dornase alfa, alpha 1-antitrypsin, heparin, dextran, capreomycin, vancomycin, meropenem, ciprofloxacin, piperacillin, and rifampicin; mucolytic agents for the treatment of COPD and cystic fibrosis, eg N-acetylcysteine, and ambroxol; histamine type 2 receptor antagonists; tachykinin neurokinin antagonists; triptans, eg almotriptan, rizatriptan, naratriptan, zolmitriptan, sumatritpan, eletriptan, and frovatriptan; neurological agents eg apomorphine, dronabinol, dihydroergotamine, and loxapine; antiviral agents eg foscarnet, acyclovir, famciclovir, valacyclovir, ganciclovir, cidofovir; amantadine, rimantadine; ribavirin; zanamivir and oseltamavir and pleconaril, protease inhibitors (eg ruprintrivir, indinavir, nelfinavir, ritonavir, and saquinavir), nucleoside reverse transcriptase inhibitors (eg didanosine, lamivudine, stavudine, zalcitabine, and zidovudine), and non-nucleoside reverse transcriptase inhibitors (eg nevirapine and efavirenz); α-1/α-2 adrenoceptor agonists, eg propylhexedrine, phenylephrine, phenylpropanolamine, ephedrine, pseudoephedrine, naphazoline, oxymetazoline, tetrahydrozoline, xylometazoline, tramazoline, and ethylnorepinephrine; platelet aggregation inhibitors/anti-inflammatory agents, eg bemiparin, enoxaparin, heparin; anti-infectives, eg cephalosporins, penicillins, tetracyclines, macrolides, beta-lactams, fluoroquinolones, streptomycin, sulphonamides, aminoglycosides (eg tobramycin), doripenem, pentamidine, colistimethate, and aztreonam; agents for sexual health, sexual dysfunction including premature ejaculation; eg. apomorphine, VR776, agents that acts via 5HT- and noradrenergic-mediated pathways in the brain, leuprolide, and PDE 5 inhibitors eg, sildenafil, tadalafil, and vardenafil; leukotriene modifiers, eg zileuton, fenleuton, tepoxalin, montelukast, zafirlukast, ontazolast, ablukast, pranlikast, verlukast, and iralukast; inducible nitric oxide synthase (iNOS) inhibitors; antifungals, eg amphotericin B, natamycin, and nystatin; analgesics, eg codeine, dihydromorphine, ergotamine, fentanyl, cannabinoids, and morphine; anxiolytic/antidepressive agents, eg benzodiazepines and benzodiazepine derivatives, diazepam, midazolam, chlordiazepoxide, lorazepam, oxazepam, clobazam, alprazolam, clonazepam, flurazepam, zolazepam; tryptase and elastase inhibitors; beta-2 integrin antagonists; adenosine receptor agonists or antagonists, eg adenosine 2α agonists; calcium channel blockers, eg gallopamil, and diltiazem; prostacyclin analogues, eg iloprost; endothelin-receptor antagonists, eg LU-135252; cytokine antagonists, eg chemokine antagonists and inhibitors and modifiers of cytokine synthesis including modifiers and inhibitors of the pro-inflammatory transcription factor, NFkB; interleukins and inhibitors of interleukins, eg aldesleukin; therapeutic proteins and peptides, eg insulin, insulin aspart, insulin glulisine; insulin lispro, neutral, regular and soluble insulins, isophane insulins, insulin zinc, protamine zinc insulin, insulin analogues, acylated insulin, insulin glargine, insulin detemir, glucagon, glucagon-like peptides, and exendins; enzymes, eg dornase alfa; systemically active macromolecules, eg human growth hormone, leuprolide, alpha-interferon, growth factors (eg insulin-like growth factor type 1), hormones, eg epinephrine, testosterone, and parathyroid hormone and analogues (eg Ostabolin-C); osteoporosis agents, eg bisphosphonates; anticancer agents, eg anthracyclines, doxorubicin, idarubicin, epirubicin, methotrexate, taxanes, paclitaxel, docetaxel, ciplatin, vinca alkaloids, vincristine, and 5-fluorouracil; anticoagulants, eg blood factors and blood factor constructs, eg FVIII-Fc and FIX-Fc; eg FV111-Fc; immunomodulators, eg cyclosporine, sirolimus, and tacrolimus; antiproliferative immunosuppressants, eg azathioprine, and mycophenolate mofetil; cytokines (eg interferons, interferon β, interleukins, and interleukin antagonists and inhibitors); nucleic acids; vaccines, eg flumist; anti-obesity agents; diagnostics and gene therapies. It will be clear to a person skilled in the art that, where appropriate, the medicaments may be linked to a carrier molecule or molecules and/or used in the form of prodrugs, salts, as esters, or as solvates to optimise the activity and/or stability of the medicament.

Inhalers according to the invention may also be used to deliver combinations of two or more different medicaments. Specific combinations of two medicaments which may be mentioned include combinations of steroids and β₂-agonists. Examples of such combinations are beclomethasone and formoterol; beclomethasone and salmeterol; fluticasone and formoterol; fluticasone and salmeterol; budesonide and formoterol; budesonide and salmeterol; flunisolide and formoterol; flunisolide and salmeterol; ciclesonide and salmeterol; ciclesonide and formoterol; mometasone and salmeterol; and mometasone and formoterol. Specifically inhalers according to the invention may also be used to deliver combinations of three different medicaments.

It will be clear to a person skilled in the art that, where appropriate, the medicaments may be linked to a carrier molecule or molecules and/or used in the form of prodrugs, salts, as esters, or as solvates to optimise the activity and/or stability of the medicament.

It is also envisaged that the pharmaceutical composition may comprise one or more, preferably one, anticholinergic 1, optionally in combination with a pharmaceutically acceptable excipient.

The anticholinergic 1 can be selected from the group consisting of

a) tiotropium salts 1a,
b) compounds of formula 1c

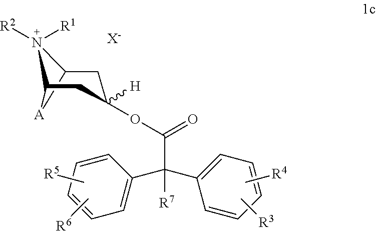

wherein
A denotes a double-bonded group selected from among

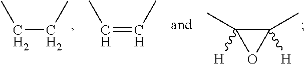

X⁻ denotes an anion with a single negative charge, preferably an anion selected from the group consisting of fluoride, chloride, bromide, iodide, sulphate, phosphate, methanesulphonate, nitrate, maleate, acetate, citrate, fumarate, tartrate, oxalate, succinate, benzoate and p-toluenesulphonate,
R¹ and R² which may be identical or different denote a group selected from among methyl, ethyl, n-propyl and iso-propyl, which may optionally be substituted by hydroxy or fluorine, preferably =substituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, -0-CH₂—F, -0-CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, -Q-COCF₃, -Q-COCF₃, fluorine, chlorine or bromine;
c) compounds of formula 1d

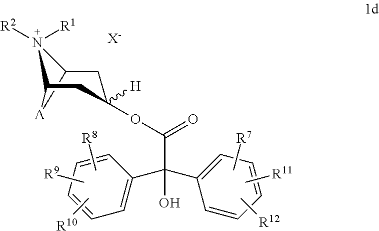

wherein

A, X⁻, R¹ and R² may have the meanings as mentioned hereinbefore and wherein R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, with the proviso that at least one of the groups R⁷, R⁸, R⁹, R¹⁰, R¹¹ and R¹² is not hydrogen,

d) compounds of formula 1e

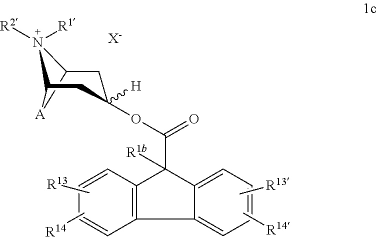

wherein A and X⁻ may have the meanings as mentioned hereinbefore, and wherein R¹⁵ denotes hydrogen, hydroxy, methyl, ethyl, —CF₃, CHF₂ or fluorine;
R^{1′} and R^{2′} which may be identical or different denote C₁-C₅-alkyl which may optionally be substituted by C₃-C₆-cycloalkyl, hydroxy or halogen, or
R^{1′} and R^{2′} together denote a —C₃-C₅-alkylene-bridge;
R¹³, R¹⁴, R^{13′} and R^{14′} which may be identical or different denote hydrogen, —C₁-C₄-alkyl, —C₁-C₄-alkyloxy, hydroxy, —CF₃, —CHF₂, CN, NO₂ or halogen,
e) compounds of formula 1f

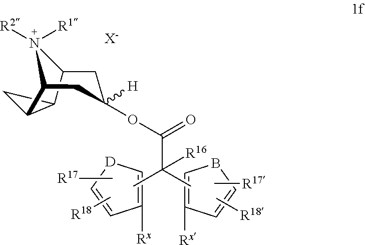

wherein X⁻ may have the meanings as mentioned hereinbefore, and wherein
D and B which may be identical or different, preferably identical, denote —O, —S, —NH, —CH₂, —CH═CH, or —N(C₁-C₄-alkyl)-;
R¹⁶ denotes hydrogen, hydroxy, —C₁-C₄-alkyl, —C₁-C₄-alkyloxy, —C₁-C₄-alkylene-Halogen, —O—C₁-C₄-alkylene-halogen, —C₁-C₄-alkylene-OH, —CF₃, CHF₂, —C₁-C₄-alkylene-C₁-C₄ alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or
R^{1″} and R^{2″} together denote a —C₃-C₅-alkylene bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂ or halogen;
R^{x} and R^{x′} which may be identical or different, denote hydrogen, C₁-C₄-alkyl, C₁-C₄-alkyloxy, hydroxy, —CF₃, —CHF₂, CN, NO₂ or halogen or
R^{x} and R^{x′} together denote a single bond or a bridging group selected from among the bridges —O, —S, —NH, —CH₂, —CH₂—CH₂—, —N(C₁-C₄-alkyl), —CH(C₁-C₄-alkyl)- and —C(C₁-C₄-alkyl)₂, and
f) compounds of formula 1g

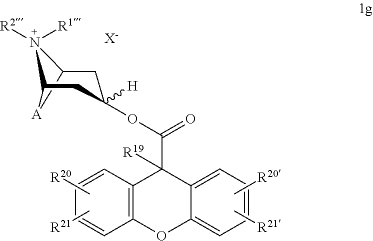

wherein X⁻ may have the meanings as mentioned hereinbefore, and wherein A′ denotes a double-bonded group selected from among

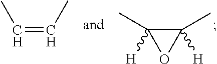

R¹⁹ denotes hydroxy, methyl, hydroxymethyl, ethyl, —CF₃, CHF₂ or fluorine;
R^{1″′} and R^{2″′} which may be identical or different denote C₁-C₅-alkyl which may optionally be substituted by C₃-C₆-cycloalkyl, hydroxy or halogen, or
R^{1″′} and R^{2″′} together denote a —C₃-C₅-alkylene-bridge;
R²⁰, R²¹, R^{20′} and R^{21′} which may be identical or different denote hydrogen, —C₁-C₄-alkyl, —C₁-C₄-alkyloxy, hydroxy, —CF₃, —CHF₂, CN, NO₂ or halogen.

The compounds of formula 1c are known in the art (WO 02/32899).

In a preferred embodiment of the invention the method comprises administration of compounds of formula 1c, wherein

X⁻ denotes bromide;
R¹ and R² which may be identical or different denote a group selected from methyl and ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine, optionally together with a pharmaceutically acceptable excipient.

Of particular importance are compounds of general formula 1c, wherein A denotes a double-bonded group selected from among

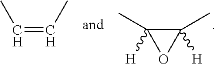

The compounds of formula 1c, may optionally be administered in the form of the individual optical isomers, mixtures of the individual enantiomers or racemates thereof.

Of particular importance within a method according to the invention are the following compounds of formula 1c:
tropenol 2,2-diphenylpropionic acid ester methobromide,
scopine 2,2-diphenylpropionic acid ester methobromide,
scopine 2-fluoro-2,2-diphenylacetic acid ester methobromide and
tropenol 2-fluoro-2,2-diphenylacetic acid ester methobromide.

The compounds of formula 1d are known in the art (WO 02/32898).

In a preferred embodiment of the invention the method comprises administration of compounds of formula 1d, wherein

A denotes a double-bonded group selected from among

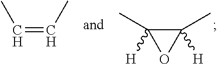

X⁻ denotes bromide;
R¹ and R² which may be identical or different denote methyl or ethyl, preferably methyl;
R⁷, R⁸, R⁹, R¹⁰, R¹¹ and R¹², which may be identical or different, denote hydrogen, fluorine, chlorine or bromine, preferably fluorine with the proviso that at least one of the groups R⁷, R⁸, R⁹, R¹⁰, R¹¹ and R¹² not hydrogen, optionally together with a pharmaceutically acceptable excipient.

Of particular importance within the method according to the invention are the following compounds of formula 1d:
tropenol 3,3′,4,4′-tetrafluorobenzilic acid ester methobromide,
scopine 3,3′,4,4′-tetrafluorobenzilic acid ester methobromide,
scopine 4,4′-difluorobenzilic acid ester methobromide,
tropenol 4,4′-difluorobenzilic acid ester methobromide,
scopine 3,3′-difluorobenzilic acid ester methobromide, and
tropenol 3,3′-difluorobenzilic acid ester methobromide.

The pharmaceutical compositions according to the invention may contain the compounds of formula 1d optionally in the form of the individual optical isomers, mixtures of the individual enantiomers or racemates thereof.

The compounds of formula 1e are known in the art (WO 03/064419).

In a preferred embodiment of the invention the method comprises administration of compounds of formula 1e, wherein

A denotes a double-bonded group selected from among

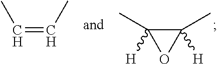

X⁻ denotes an anion selected from among chloride, bromide and methanesulphonate, preferably bromide;
R¹⁵ denotes hydroxy, methyl or fluorine, preferably methyl or hydroxy;
R^{1′} and R^{2′} which may be identical or different represent methyl or ethyl, preferably methyl;
R¹³, R¹⁴, R^{13′} and R^{14′} which may be identical or different represent hydrogen, —CF₃, —CHF₂ or fluorine, preferably hydrogen or fluorine, optionally together with a pharmaceutically acceptable excipient.

In another preferred embodiment of the invention the method comprises administration of compounds of formula 1e, wherein

A denotes a double-bonded group selected from among

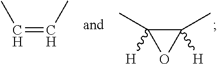

X⁻ denotes bromide;
R¹⁵ denotes hydroxy or methyl, preferably methyl;
R^{1′} and R^{2′} which may be identical or different represent methyl or ethyl, preferably methyl;
R¹³, R¹⁴, R^{13′} and R^{14′} which may be identical or different represent hydrogen or fluorine, optionally together with a pharmaceutically acceptable excipient.

Of particular importance within the method according to the invention are the following compounds of formula 1e:
tropenol 9-hydroxy-fluorene-9-carboxylate methobromide;
tropenol 9-fluoro-fluorene-9-carboxylate methobromide;
scopine 9-hydroxy-fluorene-9-carboxylate methobromide;
scopine 9-fluoro-fluorene-9-carboxylate methobromide;
tropenol 9-methyl-fluorene-9-carboxylate methobromide;
scopine 9-methyl-fluorene-9-carboxylate methobromide.

The pharmaceutical compositions according to the invention may contain the compounds of formula 1e optionally in the form of the individual optical isomers, mixtures of the individual enantiomers or racemates thereof.

The compounds of formula 1f are known in the art (WO 03/064418).

In another preferred embodiment of the invention the method comprises administration of compounds of formula 1f wherein

X⁻ denotes chloride, bromide, or methanesulphonate, preferably bromide;
D and B which may be identical or different, preferably identical, denote —O, —S, —NH or —CH═CH—;
R¹⁶ denotes hydrogen, hydroxy, —C₁-C₄-alkyl, —C₁-C₄ alkyloxy, —CF₃, —CHF₂, fluorine, chlorine or bromine;
R^{1″} and R^{2″} which may be identical or different, denote C₁-C₄-alky, which may optionally be substituted by hydroxy, fluorine, chlorine or bromine, or
R^{1″} and R^{2″} together denote a —C₃-C₄-alkylene-bridge;
R¹⁷, R¹⁸, R^{17′} and R^{18′}, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine or
R^{x} and R^{x′} together denote a single bond or a bridging group selected from among the bridges —O, —S, —NH— and —CH₂—, optionally together with a pharmaceutically acceptable excipient.

In another preferred embodiment of the invention the method comprises administration of compounds of formula 1f, wherein

X⁻ denotes chloride, bromide, or methanesulphonate, preferably bromide;
D and B which may be identical or different, preferably identical, denote —S or —

### CH═CH—;

R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen or
R^{x} and R^{x′} together denote a single bond or the bridging group —O—, optionally together with a pharmaceutically acceptable excipient.

In another preferred embodiment of the invention the method comprises administration of compounds of formula 1f wherein

X⁻ denotes bromide;
D and B denote —CH═CH—;
R¹⁶ denotes hydrogen, hydroxy or methyl;
R^{1″} and R^{2″} denote methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen or
R^{x} and R^{x′} together denote a single bond or the bridging group —O—, optionally together with a pharmaceutically acceptable excipient.

Of particular importance within the method according to the invention are the following compounds of formula 1f:
cyclopropyltropine benzilate methobromide;
cyclopropyltropine 2,2-diphenylpropionate methobromide;
cyclopropyltropine 9-hydroxy-xanthene-9-carboxylate methobromide;
cyclopropyltropine 9-methyl-fluorene-9-carboxylate methobromide;
cyclopropyltropine 9-methyl-xanthene-9-carboxylate methobromide;
cyclopropyltropine 9-hydroxy-fluorene-9-carboxylate methobromide;
cyclopropyltropine methyl 4,4′-difluorobenzilate methobromide.

The pharmaceutical compositions according to the invention may contain the compounds of formula 1f optionally in the form of the individual optical isomers, mixtures of the individual enantiomers or racemates thereof.

The compounds of formula 1g are known in the art (WO 03/064417).

In another preferred embodiment of the invention the method comprises administration of compounds of formula 1g wherein

A′ denotes a double-bonded group selected from among

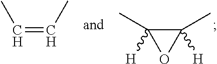

X⁻ denotes chloride, bromide or methanesulphonate, preferably bromide;
R¹⁹ denotes hydroxy or methyl;
R^{1″′} and R^{2″′} which may be identical or different represent methyl or ethyl, preferably methyl;
R²⁰, R²¹, R^{20′} and R^{21′} which may be identical or different represent hydrogen, —CF₃, —CHF₂ or fluorine, preferably hydrogen or fluorine, optionally together with a pharmaceutically acceptable excipient.

In another preferred embodiment of the invention the method comprises administration of compounds of formula 1g wherein

A′ denotes a double-bonded group selected from among

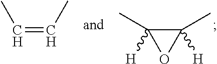

X⁻ denotes bromide;
R¹⁹ denotes hydroxy or methyl, preferably methyl;
R^{1′″} and R^{2″′} which may be identical or different represent methyl or ethyl, preferably methyl;
R³, R⁴, R^{3′} and R^{4′} which may be identical or different represent hydrogen or fluorine, optionally together with a pharmaceutically acceptable excipient.

Of particular importance within the method according to the invention are the following compounds of formula 1g:
tropenol 9-hydroxy-xanthene-9-carboxylate methobromide;
scopine 9-hydroxy-xanthene-9-carboxylate methobromide;
tropenol 9-methyl-xanthene-9-carboxylate methobromide;
scopine 9-methyl-xanthene-9-carboxylate methobromide;
tropenol 9-ethyl-xanthene-9-carboxylate methobromide;
tropenol 9-difluoromethyl-xanthene-9-carboxylate methobromide;
scopine 9-hydroxymethyl-xanthene-9-carboxylate methobromide.

The pharmaceutical compositions according to the invention may contain the compounds of formula 1g optionally in the form of the individual optical isomers, mixtures of the individual enantiomers or racemates thereof.

The alkyl groups used, unless otherwise stated, are branched and unbranched alkyl groups having 1 to 5 carbon atoms. Examples include: methyl, ethyl, propyl or butyl. The groups methyl, ethyl, propyl or butyl may optionally also be referred to by the abbreviations Me, Et, Prop or Bu. Unless otherwise stated, the definitions propyl and butyl also include all possible isomeric forms of the groups in question. Thus, for example, propyl includes n-propyl and iso-propyl, butyl includes iso-butyl, sec. butyl and tert.-butyl, etc.

The cycloalkyl groups used, unless otherwise stated, are alicyclic groups with 3 to 6 carbon atoms. These are the cyclopropyl, cyclobutyl, cyclopentyl and cyclohexyl groups. According to the invention cyclopropyl is of particular importance within the scope of the present invention.

The alkylene groups used, unless otherwise stated, are branched and unbranched double-bonded alkyl bridges with 1 to 5 carbon atoms. Examples include: methylene, ethylene, propylene or butylene.

The alkylene-halogen groups used, unless otherwise stated, are branched and unbranched double-bonded alkyl bridges with 1 to 4 carbon atoms which may be mono-, di- or trisubstituted, preferably disubstituted, by a halogen. Accordingly, unless otherwise stated, the term alkylene-OH groups denotes branched and unbranched double-bonded alkyl bridges with 1 to 4 carbon atoms which may be mono-, di- or trisubstituted, preferably monosubstituted, by a hydroxy.

The alkyloxy groups used, unless otherwise stated, are branched and unbranched alkyl groups with 1 to 5 carbon atoms which are linked via an oxygen atom. The following may be mentioned, for example: methyloxy, ethyloxy, propyloxy or butyloxy. The groups methyloxy, ethyloxy, propyloxy or butyloxy may optionally also be referred to by the abbreviations MeO, EtO, PropO or BuO. Unless otherwise stated, the definitions propyloxy and butyloxy also include all possible isomeric forms of the groups in question. Thus, for example, propyloxy includes n-propyloxy and iso-propyloxy, butyloxy includes iso-butyloxy, sec. butyloxy and tert.-butyloxy, etc. The word alkoxy may also possibly be used within the scope of the present invention instead of the word alkyloxy. The groups methyloxy, ethyloxy, propyloxy or butyloxy may optionally also be referred to as methoxy, ethoxy, propoxy or butoxy.

The alkylene-alkyloxy groups used, unless otherwise stated, are branched and unbranched double-bonded alkyl bridges with 1 to 5 carbon atoms which may be mono-, di- or trisubstituted, preferably monosubstituted, by an alkyloxy group.

The —O—CO-alkyl groups used, unless otherwise stated, are branched and unbranched alkyl groups with 1 to 4 carbon atoms which are bonded via an ester group. The alkyl groups are bonded directly to the carbonylcarbon of the ester group. The term —O—CO-alkyl-halogen group should be understood analogously. The group —O—CO—CF₃ denotes trifluoroacetate.

Within the scope of the present invention halogen denotes fluorine, chlorine, bromine or iodine. Unless otherwise stated, fluorine and bromine are the preferred halogens. The group CO denotes a carbonyl group.

One aspect of the invention is directed to an inhalation device, in which the plural of doses are contained in one reservoir. In another aspect of the invention, the inhalation device comprises the plural of doses in a multi-dose blister pack. In another aspect of the invention the inhalation device comprises the multi-dose blister pack in form of blister strip.

The inhalation device according to the invention comprises the compounds of formula 1 preferably in admixture with a pharmaceutically acceptable excipient to form a powder mixture. The following pharmaceutically acceptable excipients may be used to prepare these inhalable powder mixtures according to the invention: monosaccharides (e.g. glucose or arabinose), disaccharides (e.g. lactose, saccharose, maltose, trehalose), oligo- and polysaccharides (e.g. dextran), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose and trehalose are the particularly preferred excipients, while lactose, preferably in form of its monohydrate or anhydrate is most particularly preferred.

The compounds of formula 1 may be used in the form of their racemates, enantiomers or mixtures thereof. The separation of enantiomers from the racemates may be carried out using methods known in the art (e.g. by chromatography on chiral phases, etc.).

Optionally, the inhalation device according to the invention contains plural of doses of a medicament in powder form that contains, beside one compound of formula 1, another active ingredient.

Preferably the additional active ingredient is a beta₂ agonists 2 which is selected from the group consisting of albuterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-0X0-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts and the hydrates thereof.

According to the instant invention more preferred beta₂ agonists 2 are selected from the group consisting of bambuterol, bitolterol, carbuterol, clenbuterol, fenoterol, formoterol, hexoprenaline, ibuterol, pirbuterol, procaterol, reproterol, salmeterol, sulphonterol, terbutaline, tolubuterol, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-0X0-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts and the hydrates thereof.

More preferably, the betamimetics 2 used as within the compositions according to the invention are selected from among fenoterol, formoterol, salmeterol, 3-(4-{6-[2-Hydroxy-2-(4-hydroxy-3-hydroxymethyl-phenyl)-ethylamino]-hexyloxy}-butyl)-benzenesulfoneamide, 5-[2-(5,6-Diethyl-indan-2-ylamino)-1-hydroxy-ethyl]-8-hydroxy-1H-quinolin-2-one, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, optionally in the form of the racemates, the enantiomers, the diastereomers and optionally the pharmacologically acceptable acid addition salts thereof, and the hydrates thereof. Of the betamimetics mentioned above the compounds formoterol, salmeterol, 3-(4-{6-[2-Hydroxy-2-(4-hydroxy-3-hydroxymethyl-phenyl)-ethylamino]-hexyloxy}-butyl)-benzenesulfoneamide, and 5-[2-(5,6-Diethyl-indan-2-ylamino)-1-hydroxy-ethyl]-8-hydroxy-1H-quinolin-2-one are particularly preferred, optionally in the form of the racemates, the enantiomers, the diastereomers and optionally the pharmacologically acceptable acid addition salts thereof, and the hydrates thereof. Of the betamimetics mentioned above the compounds formoterol and salmeterol are particularly preferred, optionally in the form of the racemates, the enantiomers, the diastereomers and optionally the pharmacologically acceptable acid addition salts thereof, and the hydrates thereof.

Examples of pharmacologically acceptable acid addition salts of the betamimetics 2 according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2,4-difluorophenyl)salicylic acid or maleic acid. If desired, mixtures of the abovementioned acids may also be used to prepare the salts 2.

According to the invention, the salts of the betamimetics 2 selected from among the hydrochloride, hydrobromide, sulphate, phosphate, fumarate, methanesulphonate, 4-phenylcinnamate, 5-(2,4-difluorophenyl)salicylate, maleate and xinafoate are preferred. Particularly preferred are the salts of 2 in the case of salmeterol selected from among the hydrochloride, sulphate, 4-phenylcinnamate, 5-(2,4-difluorophenyl)salicylate and xinafoate, of which the 4-phenylcinnamate, 5-(2,4-difluorophenyl)salicylate and especially xinafoate are particularly important. Particularly preferred are the salts of 2 in the case of formoterol selected from the hydrochloride, sulphate and fumarate, of which the hydrochloride and fumarate are particularly preferred. Of exceptional importance according to the invention is formoterol fumarate.

Salts of salmeterol, formoterol, 3-(4-{6-[2-Hydroxy-2-(4-hydroxy-3-hydroxymethyl-phenyl)-ethylamino]-hexyloxy}-butyl)-benzenesulfoneamide, and 5-[2-(5,6-Diethyl-indan-2-ylamino)-1-hydroxy-ethyl]-8-hydroxy-1H-quinolin-2-one, are preferably used as the betamimetics 2 according to the invention. Of particular importance according to the invention are salmeterol and formoterol salts. Any reference to the term betamimetics 2 also includes a reference to the relevant enantiomers or mixtures thereof. In the pharmaceutical compositions according to the invention, the compounds 2 may be present in the form of their racemates, enantiomers or mixtures thereof. The separation of the enantiomers from the racemates may be carried out using methods known in the art (e.g. by chromatography on chiral phases, etc.) If the compounds 2 are used in the form of their enantiomers, it is particularly preferable to use the enantiomers in the R configuration at the C—OH group.

Optionally, the inhalation device according to the invention contains a plural of doses of a medicament in powder form, that contains beside one compound of formula 1a steroid 3 as another active ingredient.

In such medicament combinations the steroid 3 is preferably selected from among prednisolone, prednisone, butixocortpropionate, RPR-106541, flunisolide, beclomethasone, triamcinolone, budesonide, fluticasone, mometasone, ciclesonide, rofleponide, ST-126, dexamethasone, (S)-fluoromethyl 6α,9α-difluoro-17α-[(2-furanylcarbonyl)oxy]-11 [beta]-hydroxy-16α-methyl-3-oxo-androsta-1,4-diene-17β-carbothionate, (S)-(2-oxo-tetrahydro-furan-3S-yl)6α,9α-difluoro-l1β-hydroxy-16α-methyl-3-oxo-17α-propionyloxy-androsta-1,4-diene-17β-carbothionate, and etiprednol-dichloroacetate (BNP-166), optionally in the form of the racemates, enantiomers or diastereomers thereof and optionally in the form of the salts and derivatives thereof, the solvates and/or hydrates thereof.

In particularly preferred medicament combinations the steroid 3 is selected from the group comprising flunisolide, beclomethasone, triamcinolone, budesonide, fluticasone, mometasone, ciclesonide, rofleponide, ST-126, dexamethasone, (S)-fluoromethyl 6α,9α-difluoro-1 Ia-[(2-furanylcarbonyl)oxy]-11β-hydroxy-16α-methyl-3-oxo-androsta-1,4-diene-17β-carbothionate, (S)-(2-oxo-tetrahydro-furan-3S-yl)6α,9α-difluoro-11β-hydroxy-16α-methyl-3-oxo-17α-propionyloxy-androsta-1,4-diene-17β-carbothionate, and etiprednol-dichloroacetate, optionally in the form of the racemates, enantiomers or diastereomers thereof and optionally in the form of the salts and derivatives thereof, the solvates and/or hydrates thereof.

In particularly preferred medicament combinations the steroid 3 is selected from the group comprising budesonide, fluticasone, mometasone, ciclesonide, (S)-fluoromethyl 6α,9α-difluoro-11α-[(2-furanylcarbonyl)oxy]-11β-hydroxy-16α-methyl-3-oxo-androsta-1, A-diene-17β-carbothionate, and etiprednol-dichloroacetate, optionally in the form of the racemates, enantiomers or diastereomers thereof and optionally in the form of the salts and derivatives thereof, the solvates and/or hydrates thereof.

Any reference to steroids 3 includes a reference to any salts or derivatives, hydrates or solvates thereof which may exist. Examples of possible salts and derivatives of the steroids 3 may be: alkali metal salts, such as for example sodium or potassium salts, sulphobenzoates, phosphates, isonicotinates, acetates, propionates, dihydrogen phosphates, palmitates, pivalates or furcates.

Optionally, the inhalation device according to the invention contains a plural of doses of a medicament in powder form, that contains beside one compound of formula 1 additionally both, one of the betamimetics 2 mentioned hereinbefore and one of the steroids 3 mentioned hereinbefore.

Accordingly, in a preferred embodiment the invention relates to an inhalation device comprising a housing and a blister strip, the strip being movable to sequentially align each blister with means for opening a blister to enable a user to inhale said dose and, a spiral wound element to receive and coil the strip, wherein each blister contains a pharmaceutical composition in powder form wherein the pharmaceutical composition comprises one or more, preferably one, compound of formula 1.

In another embodiment, the invention relates to an inhalation device comprising a housing and a blister strip, the strip being movable to sequentially align each blister with means for opening a blister to enable a user to inhale said dose, the housing comprising a common chamber to receive the blister strip and a coil of breached blisters of that strip, the chamber being configured so that the coil of breached blisters occupies more of the space in the chamber initially occupied by the blister strip as more of the blisters of the strip are breached, wherein each blister contains a pharmaceutical composition in powder form wherein the pharmaceutical composition comprises one or more, preferably one, compound of formula 1.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore, but may also include a salt selected from ammonium chloride, ammonium orthophosphate, ammonium sulfate, barium chloride dihydrate, calcium lactate pentahydrate, copper sulfate pentahydrate, magnesium salicylate tetrahydrate, magnesium sulfate heptahydrate, potassium bisulfate, potassium bromide, potassium chromate, potassium dihydrogen orthophosphate, sodium acetate trihydrate, sodium bromoiridate dodecahydrate, sodium carbonate decahydrate, sodium fluoride, sodium hydrogen orthophosphate dodecahydrate, sodium metaperiodate trihydrate, sodium metaphosphate trihydrate, sodium metaphosphate hexahydrate, sodium sulfite heptahydrate, sodium sulfate heptahydrate, sodium sulfate decahydrate, sodium thiosulfate pentahydrate, zinc sulfate heptahydrate and combinations thereof. Preferably the salt is in the amorphous or anhydrous crystalline state.

Finally, in order to prepare the inhalable powders according to the invention, micronised active substance I—, and optionally 2 and/or 3, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art.

For the methods of preparing the pharmaceutical compositions in powder form reference may be made to the disclosure of WO 02/30390, WO 03/017970, or WO 03/017979 for example. The disclosure of WO 02/30390, WO 03/017970, and WO 03/017979 is hereby incorporated by reference into the instant patent application in its entirety.

As an example, the pharmaceutical compositions according to the invention may be obtained by the method described below.

First, the excipient and the active substance are placed in a suitable mixing container. The active substance used has an average particle size of 0.5 to 10 μm, preferably 1 to 6 μm, most preferably 2 to 5 μm. The excipient and the active substance are preferably added using a sieve or a granulating sieve with a mesh size of 0.1 to 2 mm, preferably 0.3 to 1 mm, most preferably 0.3 to 0.6 mm. Preferably, the excipient is put in first and then the active substance is added to the mixing container. During this mixing process the two components are preferably added in batches. It is particularly preferred to sieve in the two components in alternate layers. The mixing of the excipient with the active substance may take place while the two components are still being added. Preferably, however, mixing is only done once the two components have been sieved in layer by layer.

If after being chemically prepared the active substance used in the process described above is not already obtainable in a crystalline form with the particle sizes mentioned earlier, it can be ground up into the particle sizes which conform to the above-mentioned parameters (so-called micronising).

Although embodiments of the invention have been shown and described, it will be appreciated by those persons skilled in the art that the foregoing description should be regarded as a description of preferred embodiments only and that other embodiments that fall within the scope of the appended claims are considered to form part of this disclosure.

1. An inhaler for producing an inhalable aerosol of powdered medicament including an aerosolising device having a cyclone chamber of substantially circular cross-section, inlet and outlet ports at opposite ends of the chamber for the flow of drug laden air through the chamber between said ports and, a bypass air inlet for the flow of clean air into the chamber, said bypass air inlet being configured so that air entering the chamber through said inlet forms a cyclone in the chamber that interacts with the drug laden air flowing between the inlet and outlet ports.

2. An inhaler according to claim 1, wherein the bypass air inlet is configured so that bypass air enters the chamber through said bypass air inlet substantially tangential to the wall of the cyclone chamber.

3. An inhaler according to claim 1, wherein two diametrically opposed bypass air inlets are configured so that bypass air enters the chamber through each bypass air inlet substantially tangential to the wall of the cyclone chamber.

4. An inhaler according to claim 1, comprising a drug laden air flow conduit that leads to the inlet port and through which drug laden air flows prior to entry into the cyclone chamber.

5. An inhaler according to claim 4, wherein the drug laden air flow conduit is at least partially tapered in a direction towards the inlet port.

6. An inhaler according to claim 4, wherein the inlet port is offset from the longitudinal axis of the cyclone chamber.

7. An inhaler according to claim 4, comprising an impaction element positioned such that at least some drug particles in the drug laden air flow impact the impaction element.

8. An inhaler according to claim 7, wherein the impaction element is in the cyclone chamber.

9. An inhaler according to claim 8, wherein the impaction element is positioned above the inlet port such that drug particles impact the impaction element on entry into the cyclone chamber.

10. An inhaler according to claim 8, wherein the impaction element comprises a plate having an impaction surface that extends in a plane substantially at right-angles to the direction of flow of drug laden air into the chamber through the inlet port.

11. An inhaler according to claim 10, wherein the plate comprises a blade, the edges of said blade being chamfered or tapered to minimize disruption to airflow in the chamber.

12. An inhaler according to claim 5, wherein the impaction element extends radially inwardly from the side wall of the chamber above the offset inlet port so that it is located directly within the cyclonic airflow generated from bypass air entering the bypass air inlets.

13. An inhaler according to claim 12, wherein the impaction element includes an impaction surface against which drug particles impact, said impaction surface meeting the side wall of the chamber from which it extends in a smooth curve.

14. An inhaler according to claim 7, wherein the outlet port is formed from a mesh.

15. An inhaler according to claim 14, wherein the impaction element is formed in the mesh.

16. An inhaler according to claim 1, wherein the inlet port is formed from a deagglomerating mesh so that the drug laden air flows through the mesh into the cyclone chamber.

17. An inhaler according to claim 1, comprising a housing to receive a puncturable blister containing a dose of medicament for inhalation and an actuator pivotally attached to the housing, the actuator having a mouthpiece through which a dose of medicament is inhaled by a user and a blister piercing member, wherein the actuator is pivotable to cause the blister piercing member to puncture the lid of a blister, the cyclone chamber being located in the actuator.

18. An inhaler according to claim 17, wherein the housing is configured to receive a strip of blisters each containing a dose of medicament for inhalation, the actuator also being configured to sequentially move each blister into alignment with the blister piercing member so that the blister piercing member punctures the lid of an aligned blister.

19. An inhaler according to claim 17, comprising an actuator insert that locates in the mouthpiece, the cyclone chamber and the bypass air inlets being formed by said insert.

20. An inhaler according to claim 19, wherein the outlet port is formed in the insert.

21. An inhaler according to claim 19, wherein the cyclone chamber and the bypass air inlets comprise a recess in the insert and the actuator includes a plate that locates in the mouthpiece and extends over the insert to close the recess.

22. An inhaler according to claim 21, wherein the inlet port comprises an aperture in the plate for the flow of drug laden air into the cyclone chamber.

23. An inhaler according to claim 22, wherein the piercing member is attached to the actuator and extends over the plate.

24. An inhaler according to claim 23, wherein the drug laden air flow conduit is at least partially formed in the piercing member.

25. An inhaler according to claim 24, wherein the drug laden air flow conduit is formed in the piercing member and in a passageway that extend from the piercing member to the inlet port to the cyclone chamber.

26. An inhaler according to claim 24, wherein the piercing member comprises a body having a first piercing element that extends over the plate and a second piercing member that extends over the aperture in the plate, and the drug laden air flow conduit extends through the piercing member for the flow of drug laden air out of a blister and through the aperture in the plate.

27. An inhaler according to claim 22, wherein the impaction element comprises a member extending over the aperture in the plate, the member being supported by legs upstanding from the plate.

28. An inhaler according to claim 22, wherein the deagglomerating mesh is formed in the plate.

29. An inhaler according to claim 17, comprising locating pins on the actuator and cooperating lugs on the insert and the plate to position the insert and the plate within the mouthpiece.

30. An inhaler according to claim 29, wherein the piercing member locates on the pins over the insert and the plate to position the piercing member on the actuator.

31. An inhaler according to claim 17, wherein the cyclone chamber extends in an axial direction for substantially the entire height of the mouthpiece.

32. An inhaler according to claim 17, wherein the actuator comprises a diffuser at the outlet to the cyclone chamber.

33. An inhaler according to claim 1 comprising a deaggregating element located in the cyclone chamber.

34. An inhaler according to claim 33, wherein the deaggregating element comprises a plurality of vanes.

35. An inhaler according to claim 33, wherein the deaggregating element comprises a bladed element that spins in the cyclone chamber when a user inhales on the mouthpiece.

36. An inhaler according to claim 33, wherein the deaggregating element is a freely movable within the cyclone chamber.

37. (canceled)
